(19) United States
(12) Patent Application Publication
(10) Pub. No.: US 2010/0227872 A1
(43) Pub. Date: Sep. 9, 2010

(54) PYRAZINE DERIVATIVES

(76) Inventor: Mark Ian Kemp, Sandwich (GB)

Correspondence Address: PHARMACIA CORPORATION;c/o Pfizer Inc., Eastern Point Road, MS9114, Groton, CT, 06340, US

(21) Appl. No.: 12/598,308

(22) PCT Filed: Apr. 21, 2008
(86) PCT No.: PCT/IB08/01062

§ 371 Date: Mar. 26, 2010

Related U.S. Application Data
(60) Provisional application No. 60/915,752, filed on May 3, 2007.
(60) Provisional application No. 60/957,532, filed on Aug. 23, 2007.

(51) Int. Cl. A61K 31/497; C07D 413/12; C07D 403/12; A61P 29/00

(52) U.S. Cl. 514/255.05; 544/405

(57) ABSTRACT

The present invention relates to compounds of the formula

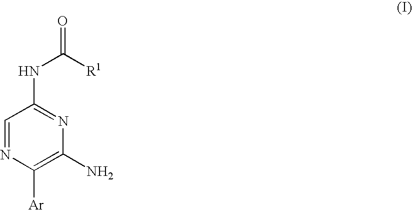

and pharmaceutically acceptable salts and solvates thereof, to processes for the preparation of, intermediates used in the preparation of, and compositions containing such compounds and the uses of such compounds for the treatment of pain.

This invention relates to pyrazine derivatives. More particularly, this invention relates to heteroaryl substituted N-[6-amino-5-aryl-pyrazin-2-yl]-carboxamide derivatives and to processes for the preparation of, intermediates used in the preparation of, compositions containing and the uses of, such derivatives.

The pyrazine derivatives of the present invention are sodium channel modulators and have a number of therapeutic applications, particularly in the treatment of pain. More particularly, the pyrazine derivatives of the invention are Na_{V1.8} modulators. Preferred pyrazine derivatives of the invention show an affinity for the Na_{V1.8} channel which is greater than their affinity for the Na_{V1.5} channel and the tetrodotoxin-sensitive sodium channels (TTX-S).

The Na_{V1.8} channel is a voltage-gated sodium channel which is expressed in nociceptors, the sensory neurones responsible for transducing painful stimuli. The rat channel and the human channel have been cloned in 1996 and 1998 respectively (Nature 1996; 379: 257-262; Pain 1998(Nov.); 78(2):107-114). The Na_{V1.8} channel was previously known as SNS (sensory neurone specific) and PN3 (peripheral nerve type 3). The Na_{V1.8} channel is atypical in that it shows resistance to the blocking effects of the puffer fish toxin tetrodotoxin and it is believed to underlie the slow-voltage-gated and tetrodotoxin-resistant (TTX-R) sodium currents recorded from dorsal root ganglion neurones. The closest molecular relative to the Na_{V1.8} channel is the Na_{V1.5} channel, which is the cardiac sodium channel, with which it shares approximately 60% homology. The Na_{V1.8} channel is expressed most highly in the ‘small cells’ of the dorsal root ganglia (DRG). These are thought to be the C- and A-delta cells which are the putative polymodal nociceptors, or pain sensors. Under normal conditions, the Na_{V1.8} channel is not expressed anywhere other than subpopulations of ORG neurones. The Na_{V1.8} channels are thought to contribute to the process of DRG sensitisation and also to hyperexcitability due to nerve injury. Inhibitory modulation of the Na_{V1.8} channels is aimed at reducing the excitability of nociceptors, by preventing them from contributing to the excitatory process.

Studies have shown that Na_{V1.8} knock-out leads to a blunted pain phenotype, mostly to inflammatory challenges (A. N. Akopian et al., Nat. Neurosci. 1999; 2; 541-548) and that Na_{V1.8} knockdown reduces pain behaviours, in this case neuropathic pain (J. Lai et al., Pain, 2002(January); 95(1-2): 143-152). Coward et al. and Yiangou et al., have shown that Na_{V1.8} appears to be expressed in pain conditions (Pain. 2000(March); 85(1-2): 41-50 and FEBS Lett. 2000(Feb. 11); 467(2-3): 249-252).

The Na_{V1.8} channel has also been shown to be expressed in structures relating to the back and tooth pulp and there is evidence for a role in causalgia, inflammatory bowel conditions and multiple sclerosis (Bucknill et al., Spine. 2002(Jan. 15); 27(2)135-140: Shembalker et al., Eur J Pain. 2001; 5(3): 319-323: Laird et al., J. Neurosci. 2002(October 1); 22(19): 8352-8356: Black et al., Neuroreport. 1999(Apr. 6); 10(5): 913-918 and Proc. Natl. Acad. Sci. USA 2000: 97:11598-11602).

Several sodium channel modulators are known for use as anticonvulsants or antidepressants, such as carbamazepine, amitriptyline, lamotrigine and riluzole, all of which target brain tetradotoxin-sensitive (TTX-S) sodium channels. Such TTX-S agents suffer from dose-limiting side effects, including dizziness, ataxia and somnolence, primarily due to action at TTX-S channels in the brain.

WO-A-03/051366 discusses protein kinase inhibitors useful for the treatment of cancer. WO-A-03/45924 discusses CRF, antagonists useful for the treatment of CNS-related disorders. WO-A-98/38174 discusses pyrazine derivatives which are stated to act as sodium channel blockers.

It is an objective of the invention to provide new Na_{V1.8} channel modulators that are good drug candidates. Preferred compounds should bind potently to the Na_{V1.8} channel whilst showing little affinity for other sodium channels, particularly the Na_{V1.5} channel and the TTX-S channels, and show functional activity as Na_{V1.8} channel modulators. They should be well absorbed from the gastrointestinal tract, be metabolically stable and possess favourable pharmacokinetic properties. They should be non-toxic and demonstrate few side-effects. Furthermore, the ideal drug candidate will exist in a physical form that is stable, non-hygroscopic and easily formulated.

Preferred pyrazine derivatives of the present invention are selective for the Na_{V1.8} channel over the Na_{V1.5} channel and the tetradotoxin-sensitive (TTX-S) sodium channels, leading to improvements in the side-effect profile.

The pyrazine derivatives of the present invention are therefore potentially useful in the treatment of a wide range of disorders, particularly pain, acute pain, chronic pain, neuropathic pain, inflammatory pain, visceral pain, nociceptive pain including post-surgical pain, and mixed pain types involving the viscera, gastrointestinal tract, cranial structures, musculoskeletal system, spine, urogenital system, cardiovascular system and CNS, including cancer pain, back and orofacial pain.

Other conditions that may be treated with the pyrazine derivatives of the present invention include multiple sclerosis, neurodegenerative disorders, irritable bowel syndrome, osteoarthritis, rheumatoid arthritis, neuropathological disorders, functional bowel disorders, inflammatory bowel diseases, pain associated with dysmenorrhea, pelvic pain, cystitis, pancreatitis, migraine, cluster and tension headaches, diabetic neuropathy, peripheral neuropathic pain, sciatica, fibromyalgia, causalgia, and conditions of lower urinary tract dysfunction.

The invention provides a pyrazine derivative of the formula (I):

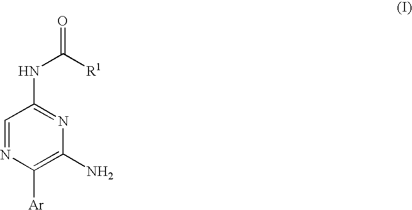

or a pharmaceutically acceptable salt or solvate thereof; wherein Ar is

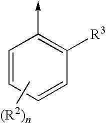

wherein→indicates the point of attachment to the pyrazine ring;
each R² is independently selected from (C₁-C₄)alkyl, (C₁-C₄)alkoxy, halo(C₁-C₄)alkyl, halo(C₁-C₄)alkoxy, cyano and halo;
n is 0 to 4;

### R³ is CF₃ or OCF₃;

R¹ is a 5-membered heteroaryl group selected from

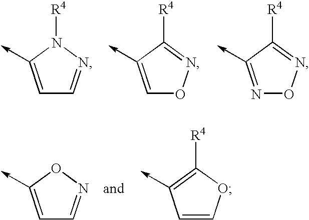

wherein→indicates the point of attachment to the carbonyl moiety; and
R⁴ is hydrogen, (C₁-C₄)alkyl, halo(C₁-C₄)alkyl, or (C₁-C₄)alkoxy(C₁-C₄)alkyl.

In the above definitions, halo means fluoro, chloro, bromo or iodo. Alkyl, and alkoxy groups, containing the requisite number of carbon atoms, can be unbranched or branched. Examples of alkyl include methyl, ethyl, propyl (n-propyl and i-propyl), and butyl (n-butyl, i-butyl, sec-butyl and t-butyl). Examples of alkoxy include methoxy, ethoxy, propoxy (n-propoxy and i-propoxy) and butoxy (n-butoxy, i-butoxy, sec-butoxy and t-butoxy). Examples of haloalkyl include trifluoromethyl. Examples of haloalkoxy include trifluoromethoxy.

In a preferred aspect (A), the invention provides a pyrazine derivative of the formula (I), or a pharmaceutically acceptable salt or solvate thereof, wherein R¹ is

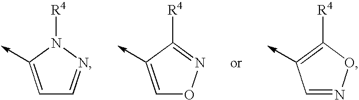

and Ar and R⁴ are as defined above.

In a preferred aspect (B), the invention provides a pyrazine derivative of the formula (I), or a pharmaceutically acceptable salt or solvate thereof, wherein Ar is as defined above, R¹ is as defined above, either in its broadest aspect or in a preferred aspect under (A); and R⁴ is (C₁-C₄)alkyl, halo(C₁-C₄)alkyl, or (C₁-C₄)alkoxy(C₁-C₄)alkyl; more preferably, R⁴ is methyl, ethyl, propyl, trifluoromethyl or methoxymethyl.

In a preferred aspect (C), the invention provides a pyrazine derivative of the formula (I), or a pharmaceutically acceptable salt or solvate thereof, wherein Ar, R¹ and R⁴ are as defined above, either in their broadest aspects or in a preferred aspect under (A) or (B), and each R² is independently selected from halo and (C₁-C₄)alkoxy; more preferably, each R² is independently selected from chloro, fluoro and ethoxy.

In a preferred aspect (D), the invention provides a pyrazine derivative of the formula (I), or a pharmaceutically acceptable salt or solvate thereof, wherein Ar, R¹, R² and R⁴ are as defined above, either in their broadest aspects or in a preferred aspect under (A), (B) or (C), and n is 0, 1, 2 or 3; more preferably, n is 0, 1 or 2.

Specific preferred pyrazine derivatives according to the invention are those listed in the Examples section below and the pharmaceutically acceptable salts and solvates thereof.

The compounds of formula (I), being Na_{V1.8} channel modulators, are potentially useful in the treatment of a range of disorders. The treatment of pain, particularly chronic, inflammatory, neuropathic, nociceptive and visceral pain, is a preferred use.

Physiological pain is an important protective mechanism designed to warn of danger from potentially injurious stimuli from the external environment. The system operates through a specific set of primary sensory neurones and is activated by noxious stimuli via peripheral transducing mechanisms (see Milian, 1999, Prog. Neurobiol., 57, 1-164 for a review). These sensory fibres are known as nociceptors and are characteristically small diameter axons with slow conduction velocities. Nociceptors encode the intensity, duration and quality of noxious stimulus and by virtue of their topographically organised projection to the spinal cord, the location of the stimulus. The nociceptors are found on nociceptive nerve fibres of which there are two main types, A-delta fibres (myelinated) and C fibres (non-myelinated). The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g. painful diabetic neuropathy, postherpetic neuralgia), carpal tunnel syndrome, back pain, headache, cancer pain, arthritic pain and chronic post-surgical pain.

When a substantial injury occurs to body tissue, via disease or trauma, the characteristics of nociceptor activation are altered and there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury. This injury often leads to abnormalities in sensory nerve fibres associated with maladaptation and aberrant activity (Woolf & Salter, 2000, Science, 288, 1765-1768).

Clinical pain is present when discomfort and abnormal sensitivity feature among the patient's symptoms. Patients tend to be quite heterogeneous and may present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia—Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive, inflammatory and neuropathic pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres. Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumour related pain (e.g. bone pain, headache, facial pain or visceral pain) or pain associated with cancer therapy (e.g. postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompass the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders that cause pain include functional bowel disorder (FBD) and inflammatory bowel disease (IBD). These GI disorders include a wide range of disease states that are currently only moderately controlled, including, in respect of FBD, gastro-esophageal reflux, dyspepsia, irritable bowel syndrome (IBS) and functional abdominal pain syndrome (FAPS), and, in respect of IBD, Crohn's disease, ileitis and ulcerative colitis, all of which regularly produce visceral pain. Other types of visceral pain include the pain associated with dysmenorrhea, cystitis and pancreatitis and pelvic pain.

It should be noted that some types of pain have multiple aetiologies and thus can be classified in more than one area, e.g. back pain and cancer pain have both nociceptive and neuropathic components.

Other types of pain include:
pain resulting from musculo-skeletal disorders, including myalgia, fibromyalgia, spondylitis, sero-negative (non-rheumatoid) arthropathies, non-articular rheumatism, dystrophinopathy, glycogenolysis, polymyositis and pyomyositis;
heart and vascular pain, including pain caused by angina, myocardical infarction, mitral stenosis, pericarditis, Raynaud's phenomenon, scleredoma and skeletal muscle ischemia;
head pain, such as migraine (including migraine with aura and migraine without aura), cluster headache, tension-type headache mixed headache and headache associated with vascular disorders; and
orofacial pain, including dental pain, otic pain, burning mouth syndrome and temporomandibular myofascial pain.

The pyrazine derivatives of formula (I) are also expected to be useful in the treatment of multiple sclerosis.

The invention also relates to therapeutic use of the pyrazine derivatives of formula (I) as agents for treating or relieving the symptoms of neurodegenerative disorders. Such neurodegenerative disorders include, for example, Alzheimer's disease, Huntington's disease, Parkinson's disease, and Amyotrophic Lateral Sclerosis. The present invention also covers treating neurodegenerative disorders termed acute brain injury. These include but are not limited to: stroke, head trauma, and asphyxia. Stroke refers to a cerebral vascular disease and may also be referred to as a cerebral vascular accident (CVA) and includes acute thromboembolic stroke. Stroke includes both focal and global ischemia. Also, included are transient cerebral ischemic attacks and other cerebral vascular problems accompanied by cerebral ischemia. These vascular disorders may occur in a patient undergoing carotid endarterectomy specifically or other cerebrovascular or vascular surgical procedures in general, or diagnostic vascular procedures including cerebral angiography and the like. Other incidents are head trauma, spinal cord trauma, or injury from general anoxia, hypoxia, hypoglycemia, hypotension as well as similar injuries seen during procedures from embole, hyperfusion, and hypoxia. The instant invention would be useful in a range of incidents, for example, during cardiac bypass surgery, in incidents of intracranial hemorrhage, in perinatal asphyxia, in cardiac arrest, and status epilepticus. A skilled physician will be able to determine the appropriate situation in which sublects are susceptible to or at risk of, for example, stroke as well as suffering from stroke for administration by methods of the present invention.

The compounds of the present invention are useful in the treatment of conditions of lower urinary tract dysfunction including but not exclusively restricted to overactive bladder, increased daytime frequency, nocturia, urgency, urinary incontinence (any condition in which there is an involuntary leakage of urine), including stress urinary incontinence, urge urinary incontinence and mixed urinary incontinence, overactive bladder with associated urinary incontinence, enuresis, nocturnal enuresis, continuous urinary incontinence, and situational urinary incontinence such as incontinence during sexual intercourse. Activity of such compounds on lower urinary tract function, and thus their potential usefulness in treating conditions involving lower urinary tract dysfunction, can be investigated and assessed utilising a number of standard in vivo models known to those skilled in the art and frequently described in the literature (Morrison, J., et al., Neurophysiology and Neuropharmacology. In: Incontinence, Ed. Abrams, P., Cardozo, C., Khoury, S, and Wein, A. Report of the World Health Organisation Consensus Conference. Paris, France: Health Publications Ltd., 2002: 83-163; Brune M E et al. Comparison of alpha 1-adrenoceptor agonists in canine urethral pressure profilometry and abdominal leak point pressure models. J Urol. 2001, 166:1555-9).

The invention also relates to therapeutic use of the pyrazine derivatives of formula (I) as agents for treating rheumatoid arthritis. Rheumatoid arthritis (RA) is considered a chronic autoimmune and inflammatory disease producing inflamed joints, which eventually swell, become painful, and experience degradation of cartilage, bone, and ligaments of the joint. A result of RA is deformity, instability, and stiffness of the joint and scarring within the joint. The joints deteriorate at a highly variable rate. Many factors, including genetic predisposition, may influence the pattern of the disease. People with rheumatoid arthritis may have a mild course, occasional flare-ups with long periods of remission without disease, or a steadily progressive disease, which may be slow or rapid. Rheumatoid arthritis may start suddenly, with many joints becoming inflamed at the same time. More often, it starts subtly, gradually affecting different joints. Usually, the inflammation is symmetric, with joints on both sides of the body affected. Typically, the small joints in the fingers, toes, hands, feet, wrists, elbows, and ankles become inflamed first, followed by the knees and hips.

Compounds of the present invention would be useful in treating arthritis, including rheumatoid arthritis, osteoarthritis, reactive arthritis (Reiter's Syndrome), infectious arthritis, psoriatic arthritis, polyarthritis, juvenile arthritis, juvenile rheumatoid arthritis, juvenile reactive arthritis and juvenile psoriatic arthritisJoint pain, also called arthralgia, can affect one or more joints. Joint pain can be caused by many types of injuries or conditions, including rheumatoid arthritis, osteoarthritis, and bursitis (i.e., inflammation of the bursae).

Other conditions that could be treated with the pyrazine derivatives of the present invention include ankylosing spondylitis; rheumatism; gonococcal arthritis; sickle cell disease; joint infection; Lyme disease; psoriasis; polymyalgia rheumatica; hemophilia; cancer; hormonal disorder; nervous system disorder; syphilis; undifferentiated spondyloarthropathy (USpA); gout; Crohn's disease; multiple sclerosis; neurodegenerative disorders; irritable bowel syndrome; neuropathalogical disorders; functional bowel disorders; inflammatory bowel disease; pain associated with dysmenorrheal; pelvic pain; cystitis; pancreatitis; migraine; cluster and tension headaches; diabetic neuropathy; peripheral neuropathic pain; sciatica; fibromyalgia; causalgia; conditions of lower urinary tract dysfunction; myasthenia gravis; Guillain-Barre; autoimmune uveitis; autoimmune hemolytic anemia; pernicious anemia; autoimmune thrombocytopenia; temporal arteritis; anti-phospholipid syndrome; vasculitides such as Wegener's granulomatosis; Behcet's disease; psoriasis; dermatitis herpetiformis; pemphigus vulgaris; vitiligo; primary biliary cirrhosis; autoimmune hepatitis; Type 1 or immune-mediated diabetes mellitus; allergic rhinitis; sinusitis; rhinosinusitis; chronic otitis media; recurrent otitis media; allergic drug reactions; allergic insect sting reactions; allergic latex reactions; conjunctivitis; urticaria; anaphylaxis reactions; anaphylactoid reactions; atopic dermatitis; asthma; food allergies; Grave's disease; Hashimoto's thyroiditis; autoimmune oophoritis and orchitis; autoimmune disorder of the adrenal gland; systemic lupus erythematosus; scleroderma; polymyositis; dermatomyositis; ankylosing spondylitis; Sjogren's syndrome and ulcerative colitis.

The pyrazine derivatives of formula (I) are also expected to be useful in the treatment of:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis; and
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I); or) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order ('glass transition'). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order ('melting point').

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 6418), 1269-1288, by Haleblian (August 1975).

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) include references to salts, solvates, multi-component complexes and liquid crystals thereof and to solvates, multi-component complexes and liquid crystals of salts thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

As indicated, so-called ‘prodrugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃—>—CH₂OH):
(ii) where the compound of formula (I) contains an alkoxy group, an hydroxy derivative thereof (—OR—>—OH); and
(iii) where the compound of formula (I) contains a phenyl moiety, a phenol derivative thereof (-Ph—>-PhOH).

Compounds of formula (I) containing one or more asymmetric carbon atoms can exist as two or more stereoisomers. Where structural isomers are interconvertible via a low energy barrier, tautomeric isomerism ('tautomerism') can occur. This can take the form of so-called valence tautomerism in compounds which contain an aromatic moiety. It follows that a single compound may exhibit more than one type of isomerism. Included within the scope of the present invention are all stereoisomers and tautomeric forms of the compounds of formula (I), including compounds exhibiting more than one type of isomerism, and mixtures of one or more thereof. Also included are acid addition or base salts wherein the counterion is optically active, for example, d-lactate or /-lysine, or racemic, for example, d/-tartrate or d/-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (I) should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function.

The compound of formula (I) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (I) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(d/-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as /-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff”. The overall daily dose may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 0.1 mg to 1000 mg depending, of course, on the mode of administration. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

A Na_{V1.8} channel modulator may be usefully combined with another pharmacologically active compound, or with two or more other pharmacologically active compounds, particularly in the treatment of pain. For example, a Na_{V1.8} channel modulator, particularly a compound of formula (I), or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)-quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3. NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-amino-5-methyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S42-[(1-iminoethyl)amino]ethyl)-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl),1,4-benzoquinone (CV-0504);
a sodium channel blocker, such as lidocaine;
a 5-HT3 antagonist, such as ondansetron;
   and the pharmaceutically acceptable salts and solvates thereof.

Such combinations offer significant advantages, including synergistic activity, in therapy.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

All of the pyrazine derivatives of the formula (I) can be prepared by the procedures described in the general methods presented below or by routine modifications thereof. The present invention also encompasses any one or more of these processes for preparing the pyrazine derivatives of formula (I), in addition to any novel intermediates used therein.

In the following general methods, Ar and R¹ are as previously defined for a pyrazine derivative of the formula (I) unless otherwise stated. Where ratios of solvents are given, the ratios are by volume.

According to a first process, compounds of formula (I) may be prepared from compounds of formula (V), as illustrated by Scheme1.

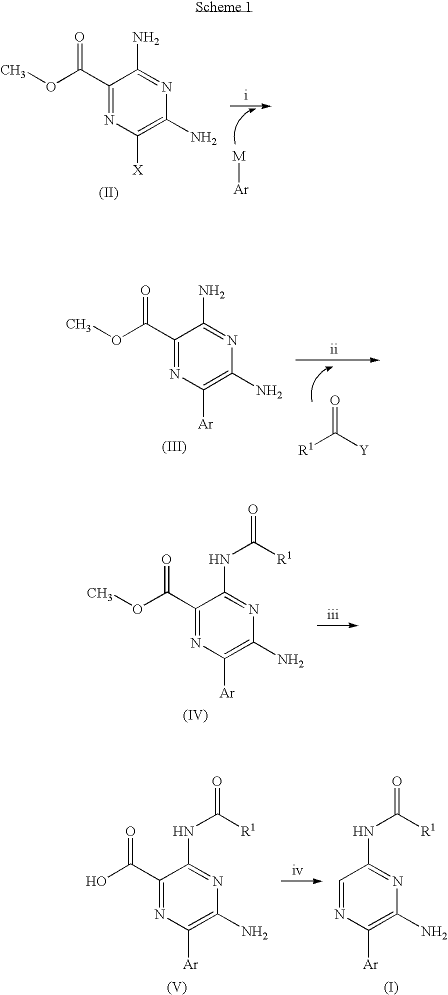

M is an optionally substituted metal or boron group suitable for cross-coupling reactions such as a trialkylstannane, dihydroxyborane, dialkoxyborane or halozinc.
X is a suitable group for cross-coupling reactions, typically Cl, Br or I
Y is a suitable leaving group, typically Cl

Compounds of formula (II) are either commercially available, in the case of the chloro derivative, or are known in the literature (J. Med. Chem. 1967, 10(1), 66-75).

Compounds of formula (III) can be prepared from compounds of formula (II) by process step (i), a cross-coupling reaction, with ArM, in the presence of a suitable catalyst system, (e.g. palladium or nickel), and base. Typically ‘Suzuki’ conditions are used, comprising 1.2-3 equivalents of boronic acid, base and 0.01-0.25 equivalents of a palladium catalyst with phosphine based ligands in an organic solvent at a temperature of from 50° C. to 100° C. Preferred conditions comprise 2 equivalents of boronic acid, 1 equivalent of Cs₂CO₃ and 0.1 equivalents Pd(PPh₃)₄ in 2:1 1.4-dioxane/water at 80° C.

Compounds of formula (IV) can be prepared from compounds of formula (III) according to process step (ii), an amide coupling using an acid chloride or a carboxylic acid activated by a suitable agent, optionally in the presence of a catalyst, in a suitable solvent. Typical conditions comprise acid chloride and an amine of formula with an excess of a suitable organic base, such as Et₃N, lutidine or pyridine, in a suitable solvent, at a temperature of from room temperature to 80° C. Preferred conditions comprise 1.5 equivalents acid chloride in pyridine at 60° C., or with 1.5 equivalents lutidine in acetonitrile at room temperature.

Compounds of formula (V) can be prepared from compounds of formula (IV) according to process step (iii), an ester hydrolysis reaction under basic, or acidic conditions. Typical conditions are base mediated, using an alkali metal base such as LiOH, NaOH, KOH or K₂CO₃ in the presence of water and a suitable solvent at a temperature of from room temperature to 100° C. Preferred conditions comprise 3 equivalents of LiOH.H₂O in 3:1 CH₃OH/H₂O at 75° C.

Compounds of formula (I) can be prepared from compounds of formula (V) by decarboxylation under basic or acidic conditions requiring a temperature of from 50° C. to 150° C. (process step (iv)). Typical conditions comprise an excess of aqueous acid in a suitable organic solvent at a temperature of from 50(2C to 100° C. Preferably the decarboxylation step is carried at reflux in 2:1 1N aqueous HCl/1,4-dioxane.

According to a second process, compounds of formula (I) may be prepared from compounds of formula (VII), as illustrated by Scheme 2.

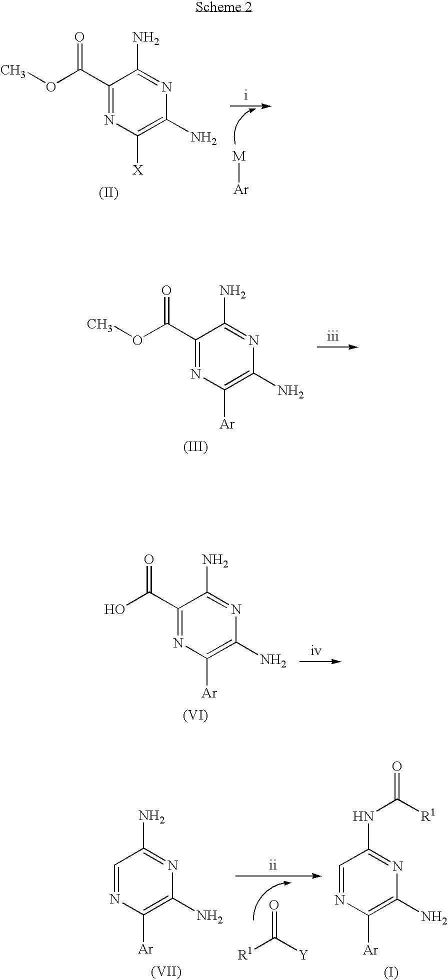

wherein M, X and Y are as defined for Scheme 1.

Compounds of formula (III) can be prepared from compounds of formula (II) according to process step (i) as described above for Scheme 1.

Compounds of formula (VI) can be prepared from compounds of formula (III) by ester hydrolysis according to process step (iii) as described above for Scheme 1.

Compounds of formula (VII) can be prepared from compounds of formula (VI) by decarboxylation according to process step (iv) as described above for Scheme 1.

Compounds of formula (I) can be prepared from compounds of formula (VIII) by an amide coupling reaction according to process step (ii) as described above for Scheme 1.

Compounds of formula (VII) may also be prepared according to a third process as described in WO-A-98/3817 (Scheme 3).

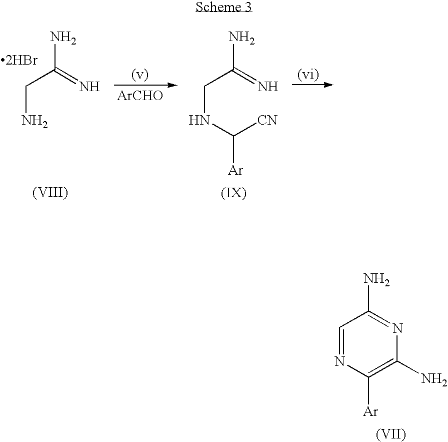

Compounds of formula (IX) may be prepared, according to process step (v), by reacting compounds of formula (VIII) or a salt thereof, for example aminoacetamidine, with compounds of formula ArCHO in the presence of a cyanide source, for example potassium cyanide.

Compounds of formula (VII) may be prepared by cyclisation and oxidation of a compound of formula (IX) in the presence of lithium hydroxide in a suitable alcoholic solvent such as methanol, with the reaction open to the air for oxidation.

According to a fourth process, compounds of formula (I) may be prepared from compounds of formula (XII), as illustrated by Scheme 4.

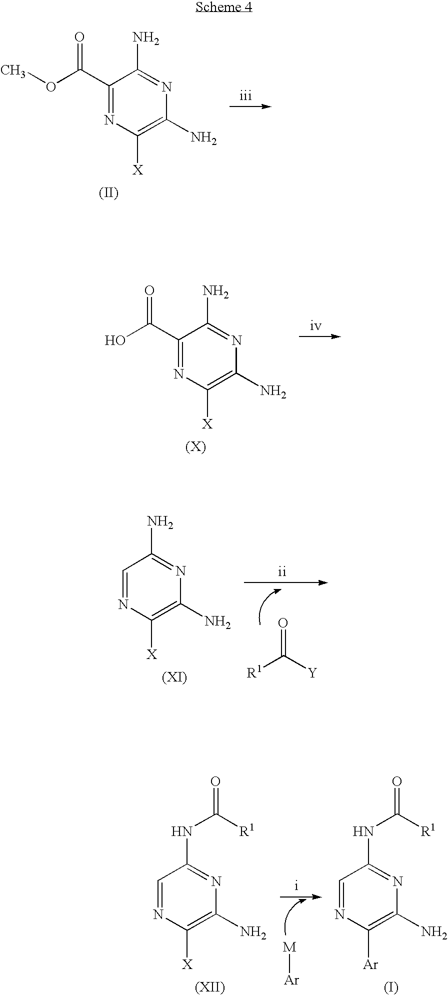

M, X and Y are as defined for Scheme 1.

Compounds of formula (X) can be prepared from compounds of formula (II) by ester hydrolysis according to process step (iii) as described above for Scheme 1.

Compounds of formula (XI) can be prepared from compounds of formula (X) by decarboxylation according to process step (iv) as described above for Scheme 1.

Compounds of formula (XII) can be prepared from compounds of formula (XI) by an amide coupling reaction according to process step (ii) as described above for Scheme 1.

Compounds of formula (I) can be prepared from compounds of formula (XII) by a cross-coupling reaction according to process step (i) as described above for Scheme 1.

Compounds of formula (XI) may alternatively be prepared from compounds of formula (XIII), as illustrated by Scheme 5.

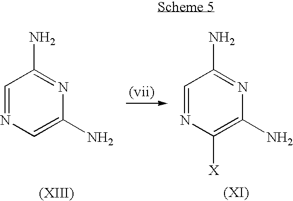

wherein X is a halogen atom.
2,6-Diaminopyrazine may be prepared as described in J. Chem. Soc. Perkin Trans. 1: Organic and Bio-Organic Chemistry (1972-1999) 1973, 6, 606.

Compounds of formula (XI) may be prepared by an electrophilic halogenation reaction according to reaction step (vii). Typical conditions comprise reaction of 2,6-diaminopyrazine with a halogen, optionally in the presence of a catalyst, e.g. iodine and silver acetate or bromine in a suitable solvent. Preferred conditions comprise bromine in acetic acid at room temperature.

Alternatively, compounds of formula (XII) may be prepared from compounds of formula (XIV), as illustrated by Scheme 6.

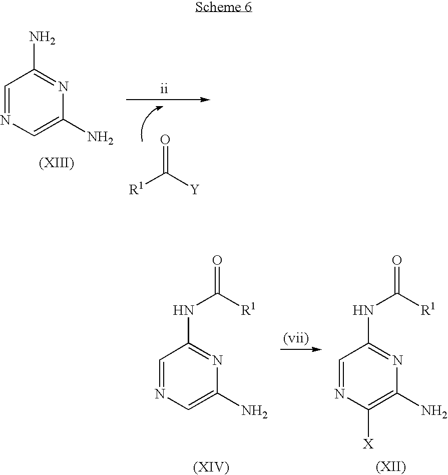

wherein Y is as defined for Scheme 1; and
X is a halogen atom.

Compounds of formula (XIV) may be prepared from compounds of formula (XIII) by an amide coupling reaction according to process step (ii) as described for Scheme 1.

Compounds of formula (XII) may be prepared from compounds of formula (XIV) by an electrophilic halogenation reaction according to process step (yip as described for Scheme 5.

Referring to the general methods above, it will be readily understood to the skilled person that where protecting groups are present, these will be generally interchangeable with other protecting groups of a similar nature, e.g. where an amine is described as being protected with a tert-butoxycarbonyl group, this may be readily interchanged with any suitable amine protecting group. Suitable protecting groups are described in ‘Protective Groups in Organic Synthesis’ by T. Greene and P. Wuts ord edition, 1999, John Wiley and Sons).

The present invention also relates to novel intermediate compounds as defined above, all salts, solvates and complexes thereof and all solvates and complexes of salts thereof as defined hereinbefore for pyrazine derivatives of formula V). The invention includes all polymorphs of the aforementioned species and crystal habits thereof.

When preparing pyrazine derivatives of formula (I) in accordance with the invention, it is open to a person skilled in the art to routinely select the best order of steps with which to synthesise the intermediates, and to choose the form of the intermediate compounds which provides the best combination of features for this purpose. Such features include the melting point, solubility, processability and yield of the intermediate form and the resulting ease with which the product may be purified on isolation.

The skilled person may undertake the synthetic steps described above in any suitable order to arrive at the compounds of formula (I).

The invention is illustrated by the following representative Examples.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The mass spectra (MS) were recorded using either electrospray ionisation (ESI) or atmospheric pressure chemical ionisation (APCI). The following abbreviations have been used for common solvents: CDCl₃, deuterochloroform; D₆-DMSO, deuterodimethylsulphoxide; CD₃OD, deuteromethanol; THF, tetrahydrofuran. LCMS indicates liquid chromatography mass spectrometry (Rₜ=retention time). Where ratios of solvents are given, the ratios are by volume.

Certain compounds of the Examples and Preparations were purified using Automated Preparative High Performance Liquid Chromatography (HPLC). Reversed-phase HPLC conditions were on FractionLynx systems. Samples were submitted dissolved in 1 mL of DMSO. Depending on the nature of the compounds and the results of a pre-analysis, the purification was performed under either acidic conditions or basic conditions at ambient temperature. Acidic runs were carried out on a Sunfire Prep C18 OBD column (19×50 mm, 5 μm), basic runs were carried out on a Xterra Prep MS C18 (19×50 mm, 5 μm), both from Waters. A flow rate of 18 mL/min was used with mobile phase A: water+0.1% modifier (v/v) and B: acetonitrile+0.1% modifier (v/v). For acidic runs the modifier was formic acid, for basic run the modifier was diethylamine. A Waters 2525 binary LC pump supplied a mobile phase with a composition of 5% B for 1 min then ran from 5% to 98% B over 6 min followed by a 2 min hold at 98% B.

Detection was achieved using a Waters 2487 dual wavelength absorbance detector set at 225 nm followed in series by a Polymer Labs PL-ELS 2100 detector and a Waters ZQ 2000 4 way MUX mass spectrometer in parallel. The PL 2100 ELSD was set at 30° C. with 1.6 L/min supply of Nitrogen. The Waters ZQ MS was tuned with the following parameters:
ES+ Cone voltage: 30 v Capillary: 3.20 kv
ES− Cone voltage: −30 v Capillary: −3.00 kv

### Desolvation gas: 600 L/hr

### Source Temp: 120° C.

Scan range 150-900 Da

The fraction collection was triggered by both MS and ELSD.

Quality control analysis was performed using a LCMS method orthogonal to the preparative method. Acidic runs were carried out on a Sunfire C18 (4.6×50 mm, 5 μm), basic runs were carried out on a Xterra C18 (4.6×50 mm, 5 μm), both from Waters. A flow rate of 1.50 L/min was used with mobile phase A: water+0.1% modifier (v/v) and B: acetonitrile+0.1% modifier (v/v). For acidic runs the modifier was formic acid, for basic run the modifier was diethylamine. A Waters 1525 binary LC pump ran a gradient elution from 5% to 95% B over 3 min followed by a 1 min hold at 95% B. Detection was achieved using a Waters MUX UV 2488 detector set at 225 nm followed in series by a Polymer Labs PL-ELS 2100 detector and a Waters ZQ 2000 4 way MUX mass spectrometer in parallel. The PL 2100 ELSD was set at 30° C. with 1.6 L/min supply of Nitrogen. The Waters ZQ MS was tuned with the following parameters:

ES+ Cone voltage: 25 v Capillary: 3.30 kv
ES−Cone voltage: −30 v Capillary: −2.50 kv

### Desolvation gas: 800 L/hr

### Source Temp: 150° C.

Scan range 160-900 Da

### EXAMPLE 1

### N-{6-Amino-5-[2-(trifluoromethoxy)phenyl]pyrazin-2-yl}-3-methylisoxazole-4-carboxamide

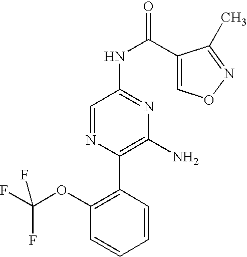

Oxalyl chloride (0.107 ml, 1.23 mmol) was added to a slurry of 3-methylisoxazole-4-carboxylic acid (0.20 g, 1.57 mmol) in dichloromethane (10 ml). Two drops dimethylformamide were added and the reaction left to stir at room temperature for 3 hours. The reaction was concentrated in vacuo and azeotroped with dichloromethane. The residue was dissolved in CH₃CN to make a 0.25M solution. 3.65 ml of the 0.25M solution of acid chloride (0.913 mmol) in CH₃CN was added to a solution of the 3-[2-(trifluoromethoxy)phenyl]pyrazine-2,6-diamine (Preparation 2, 0.235 g, 0.87 mmol) and lutidine (0.146 ml, 1.30 mmol) in CH₃CN (10 ml). The reaction was warmed to room temperature and stirred for 18 hours before concentrating in vacuo. The residue was taken up in ethyl acetate and washed with a saturated aqueous solution of NaHCO₃ before drying over MgSO₄ and concentrating in vacuo. The residue was purified by silica gel column chromatography eluting with 20:80 to 45:65 ethyl acetate:heptane to afford the title compound (0.203 g, 62% yield).

¹HNMR (d₆-DMSO): 2.44 (s, 3H), 5.96 (br s, 2H), 7.48-7.60 (m, 4H), 8.61 (s, 1H), 9.59 (s, 1H), 10.66 (br s, 1H).

### EXAMPLE 2

### N-{6-Amino-5-[2-(trifluoromethyl)phenyl]pyrazin-2-yl}-3-methylisoxazole-4-carboxamide

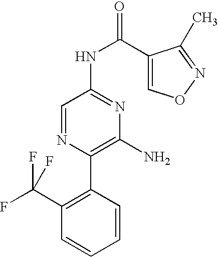

Oxalyl chloride (0.107 ml, 1.23 mmol) was added to a slurry of 3-methylisoxazole-4-carboxylic acid (0.104 g, 0.818 mmol) in dichloromethane (6 ml). One drop dimethylformamide was added and the reaction left to stir at room temperature for 4 hours. The reaction was concentrated in vacuo and azeotroped with dichloromethane. The residue was dissolved in CH₃CN to make a 0.1M solution. 2.02 ml of the 0.1M solution of acid chloride (0.202 mmol) in CH₃CN was added to a solution of the 3-[2-(trifluoromethyl)phenyl]pyrazine-2,6-diamine (Preparation 4, 0.049 g, 0.193 mmol) and lutidine (0.028 ml, 0.251 mmol) in CH₃CN (7 ml). The reaction was warmed to room temperature and stirred for 24 hours. A further 0.0216 ml lutidine (0.193 mmol) and 0.965 ml of the 0.1M acid chloride solution (0.0965 mmol) were added and the reaction stirred at room temperature for 52 hours before concentrating in vacuo. The residue was taken up in ethyl acetate and washed with a saturated aqueous solution of NaHCO₃ before drying over MgSO₄ and concentrating in vacuo. The residue was purified by silica gel column chromatography eluting with 40:60 to 66:33 ethyl acetate:heptane, followed by preparative HPLC to afford the title compound.

LCMS Rt=3.04 min

MS m/z 364 [MH]

### EXAMPLE 3

N-{6-Amino-5-[5-chloro-2-(trifluoromethoxy)phenyl]pyrazin-2-yl}-3-methylisoxazole-4-carboxamide

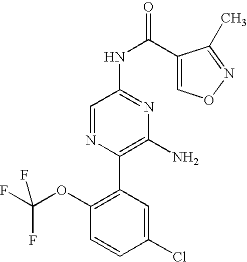

Oxalyl chloride (1.00 ml, 11.7 mmol) was added to a slurry of 3-methylisoxazole-4-carboxylic acid (0.5 g, 3.93 mmol) in dichloromethane (30 ml). Two drops dimethylformamide were added and the reaction left to stir at room temperature for 18 hours. The reaction was concentrated in vacuo. The residue was dissolved in CH₃CN to make a 1M solution. 0.175 ml of the 1M solution of acid chloride (0.175 mmol) in CH₃CN was added to a solution of the 3-[5-chloro-2-(trifluoromethyoxy)phenyl]pyrazine-2,6-diamine (Preparation 3, 0.038 g, 0.12 mmol) and lutidine (0.024 ml, 0.21 mmol) in CH₃CN (3 ml). The reaction was warmed to room temperature and stirred for 72 hours before concentrating in vacuo. The residue was partitioned between dichloromethane and a saturated aqueous solution of NaHCO₃ and separated using a phase separation cartridge. The organic layer was dried over MgSO₄ and concentrating in vacuo. The residue was purified by preparative HPLC to afford the title compound.

LCMS Rt=3.44 min

MS m/z 414 [MH]+

### EXAMPLE 4

### N-{6-Amino-5-[2-(trifluoromethoxy)phenyl]pyrazin-2-yl}-1-ethyl-1H-pyrazole-5-carboxamide

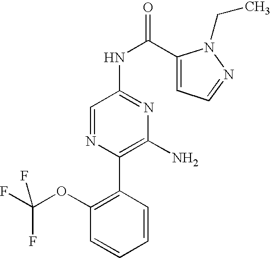

A suspension of 1-ethyl-1H-pyrazole-5-carboxylic acid (0.075 g, 0.54 mmol) in thionyl chloride (3 ml) was heated at 80° C. for 4 hours. The reaction was then concentrated in vacuo and azeotroped with dichloromethane. The residue was dissolved in CH₃CN to make a 1M solution. 0.259 ml of the 1M solution of acid chloride (0.259 mmol) in CH-₃CN was added to a solution of the 3-[2-(trifluoromethyoxy)phenyl]pyrazine-2,6-diamine (Preparation 2, 0.05 g, 0.19 mmol) and lutidine (0.031 ml, 0.282 mmol) in CH₃CN (5 ml). The reaction was warmed to room temperature and stirred for 18 hours before concentrating in vacuo. The residue was partitioned between dichloromethane and water and separated using a phase separation cartridge. The organic layer was dried over MgSO₄ and concentrating in vacuo. The residue was purified by preparative HPLC to afford the title compound.

LCMS Rt=3.20 min

MS m/z 393 [MH]+

### EXAMPLE 5

### N-{6-Amino-5-[2-(trifluoromethoxy)phenyl]pyrazin-2-v1}-1-isopropyl-1H-pyrazole-5-carboxamide

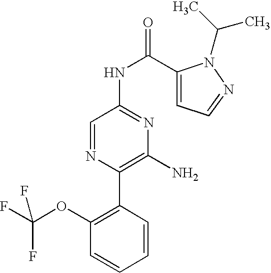

A suspension of 1-isopropyl-1H-pyrazole-5-carboxylic acid (0.05 g, 0.32 mmol) in thionyl chloride (3 ml) was heated at 80° C. for 3 hours. The reaction was then concentrated in vacuo and azeotroped with dichloromethane. The residue was dissolved in CH₃CN to make a 0.1M solution. 1.85 ml of the 0.1M solution of acid chloride (0.185 mmol) in CH₃CN was added to a solution of the 3-[2-(trifluoromethyoxy)phenyl]pyrazine-2,6-diamine (Preparation 2, 0.05 g, 0.19 mmol) and lutidine (0.021 ml, 0.185 mmol) in CH₃CN (4 ml). The reaction was warmed to room temperature and stirred for 18 hours. A further 1.35 ml of the 0.1M acid chloride solution (0.135 mmol) was added and the reaction stirred for a further 72 hours before concentrating in vacuo. The residue was partitioned between dichloromethane and water and separated using a phase separation cartridge. The organic layer was dried over MgSO₄ and concentrating in vacuo. The residue was purified by preparative HPLC to afford the title compound.

LCMS Rt=3.90 min

MS m/z 407 [MH]+

### EXAMPLE 6

### N-{6-Amino-5-[2-(trifluoromethoxy)phenyl]pyrazin-2-yl}-3-(methoxymethyl)isoxazole-4-carboxamide

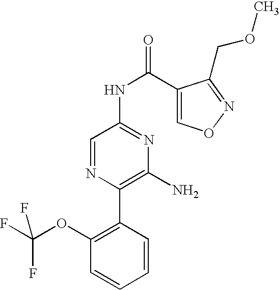

Oxalyl chloride (0.5 ml, 5.73 mmol) was added to a slurry of 3-(methoxymethyl)isoxazole-4-carboxylic acid and 3-(methoxymethyl)isoxazole-5-carboxylic acid (Preparation 7, 0.30 g, 1.91 mmol) in dichloromethane (10 ml). Two drops dimethylformamide were added and the reaction left to stir at room temperature for 2 hours. The reaction was concentrated in vacuo and azeotroped with dichloromethane. The residue was dissolved in CH₃CN to make a 1M solution. 0.32 ml of the 1M solution of acid chloride (0.056 g, 0.32 mmol) in CH₃CN was added to a solution of the 3-[(2-(trifluoromethyoxy)phenyl]pyrazine-2,6-diamine (Preparation 2, 0.08 g, 0.3 mmol) and lutidine (0.1 ml, 0.859 mmol) in CH₃CN (2 ml). The reaction was warmed to room temperature and stirred for 18 hours. A further 0.07 ml of the 1M acid chloride solution (0.07 mmol) was added and the reaction stirred for a further 72 hours before concentrating in vacuo. The residue was dissolved in ethyl acetate (60 ml) and water (30 ml) added. The aqueous layer was acidified to pH 2 with 2M hydrochloric acid. The organic layer was dried over Na₂SO₄ and concentrated in vacuo. The residue was purified by silica gel column chromatography eluting with 65:35 to 50:50 heptane:ethyl acetate to afford the title compound (0.011 g, 9% yield) ¹HNMR (CDCl₃): 3.66 (s, 3H), 4.49 (br s, 2H), 4.85 (s, 2H), 7.42-7.57 (m, 4H), 9.07 (s, 1H), 9.11 (s, 1H), 10.30 (br s, 1H).

LCMS Rt=2.95 min

MS m/z 410 [MH]+

### EXAMPLE 7

### N-{6-Amino-5-[6-chloro-2-trifluoromethoxy)phenyl]pyrazin-2-yl}-3-(methoxymethyl)isoxazole-4-carboxamide

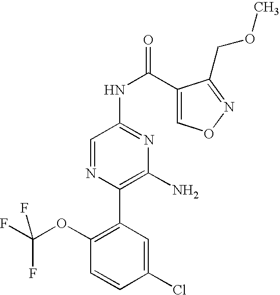

Oxalyl chloride (0.5 ml, 5.73 mmol) was added to a slurry of 3-(methoxymethyl)isoxazole-4-carboxylic acid and 3-(methoxymethyl)isoxazole-5-carboxylic acid (Preparation 7, 0.30 g, 1.91 mmol) in dichloromethane (10 ml). Two drops dimethylformamide were added and the reaction left to stir at room temperature for 2 hours. The reaction was concentrated in vacuo and azeotroped with dichloromethane. The residue was dissolved in CH₃CN to make a 1M solution. 0.2 ml of the 1M solution of acid chloride (0.035 g, 0.2 mmol) in CH₃CN was added to a solution of the 3-[5-chloro-2-(trifluoromethyoxy)phenyl]pyrazine-2,6-diamine (Preparation 3, 0.054 g, 0.18 mmol) and lutidine (0.06 ml, 0.05 mmol) in CH₃CN (1 ml). The reaction was warmed to room temperature and stirred for 3 hours. A further 0.1 ml of the 1M acid chloride solution (0.1 mmol) was added and the reaction stirred for a further 18 hours before concentrating in vacuo. The residue was dissolved in ethyl acetate (80 ml) and water (30 ml) added. The aqueous layer was acidified to pH 2 with 2M hydrochloric acid. The organic layer was dried over Na₂SO₄ and concentrated in vacuo. The residue was purified by silica gel column chromatography eluting with 65:35 to 60:40 heptane:ethyl acetate to afford the title compound (0.005 g, 6% yield) ¹HNMR (CDCl₃): 3.65 (s, 3H), 4.51 (br s, 2H), 4.85 (s, 2H), 7.35 (d, 1H), 7.46 (d, 1H), 7.57 (s, 1H), 9.07 (s, 1H), 9.11 (s, 1H), 10.34 (br s, 1H).

LCMS Rt=3.16 min

MS m/z 444 [MH]+

### EXAMPLE 8

### N-{6-Amino-5-[5-fluoro-2-(trifluoromethyl)phenyl]pyrazin-2-yl}-3-(methoxymethyl)isoxazole-4-carboxamide

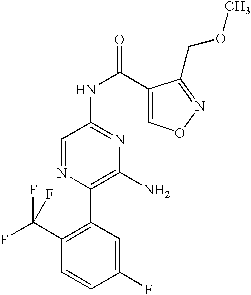

Oxalyl chloride (0.5 ml, 5.73 mmol) was added to a slurry of 3-(methoxymethyl)isoxazole-4-carboxylic acid and 3-(methoxymethyl)isoxazole-5-carboxylic acid (Preparation 7, 0.30 g, 1.91 mmol) in dichloromethane (10 ml). Two drops dimethylformamide were added and the reaction left to stir at room temperature for 2 hours. The reaction was concentrated in vacuo and azeotroped with dichloromethane. The residue was dissolved in CH₃CN to make a 1M solution. 0.12 ml of the 1M solution of acid chloride (0.021 g, 0.12 mmol) in CH₃CN was added to a solution of the 3-[5-fluoro-2-(trifluoromethyoxy)phenyl]pyrazine-2,6-diamine (Preparation 5, 0.03 g, 0.11 mmol) and lutidine (0.04 ml, 0.34 mmol) in CH₃CN (1 ml). The reaction was warmed to room temperature and stirred for 18 hours. A further 0.015 ml of the 1M acid chloride solution (0.015 mmol) was added and the reaction stirred for a further 90 hours before concentrating in vacuo. The residue was dissolved in ethyl acetate (50 ml) and water (20 ml) added. The aqueous layer was acidified to pH 2 with 2M hydrochloric acid. The organic layer was dried over Na₂SO₄ and concentrated in vacuo. The residue was purified by silica gel column chromatography eluting with 65:35 to 50:50 heptane:ethyl acetate to afford the title compound (0.003 g, 7% yield)

¹HNMR (CDCl₃): 3.65 (s, 3H), 4.33 Or s, 2H), 4.85 (s, 2H), 7.18 (m, 2H), 7.87 (m, 1H), 9.04 (s, 1H), 9.11 (s, 1H), 10.33 (br s, 1H).

LCMS Rt=3.03 min

MS m/z 412 [MH]+

### EXAMPLE 9

### N-{6-Amino-5-[2-(trifluoromethoxy)phenyl]pyrazin-2-yl}-5-(methoxymethyl)isoxazole-4-carboxamide

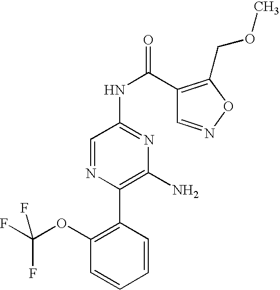

Oxalyl chloride (0.951 ml, 10.9 mmol) was added to a slurry of 5-(methoxymethyl)isoxazole-4-carboxylic acid (Preparation 11, 0.57 g, 3.63 mmol) in dichloromethane (10 ml). Two drops dimethylformamide were added and the reaction left to stir at room temperature for 3.5 hours. The reaction was concentrated in vacuo and azeotroped with dichloromethane. The residue was dissolved in CH₃CN to make a 1M solution. 0.224 ml of the 1M solution of acid chloride (0.0393 g, 0.224 mmol) in CH₃CN was added to a solution of the 3-[2-(trifluoromethyoxy)phenyl]pyrazine-2,6-diamine (Preparation 2, 0.055 g, 0.2 mmol) and lutidine (0.0284 ml, 0.244 mmol) in CH₃CN (2 ml). The reaction was warmed to room temperature and stirred for 18 hours. A further 0.15 ml of the 1M acid chloride solution (0.15 mmol) was added and the reaction stirred for a further 18 hours before concentrating in vacuo. The residue was dissolved in ethyl acetate (50 ml) and a saturated aqueous solution of NaHCO₃ (20 ml) was added. The organic layer was dried over MgSO₄ and concentrated in vacuo. The residue was purified by preparative HPLC to afford the title compound.

LCMS Rt=3.31 min

MS m/z 410 [MH]+

### EXAMPLE 10

### N-{6-amino-5-[5-fluoro-2-(trifluoromethyl)phenyl]pyrazin-2-yl}-3-methylisoxazole-4-carboxamide

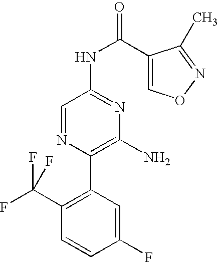

Oxalyl chloride (0.077 ml, 0.885 mmol) was added to a slurry of 3-methylisoxazote-4-carboxylic acid (0.075 g, 0.59 mmol) in dichloromethane (3 ml). Two drops dimethylformamide were added and the reaction left to stir at room temperature for 4 hours. The reaction was concentrated in vacuo and azeotroped with dichloromethane. The residue was dissolved in CH₃CN (2 ml). 0.55 ml of the solution of acid chloride (0.024 g, 0.165 mmol) in CH₃CN was added to a solution of the 3-[5-fluoro-2-(trifluoromethyl)phenyl]pyrazine-2,6-diamine (Preparation 5, 0.030 g, 0.111 mmol) and lutidine (0.019 ml, 0.176 mmol) in CH₃CN (2 ml). The reaction was warmed to room temperature and stirred for 24 hours. A further 0.365 ml of the acid chloride solution (0.016 g, 0.111 mmol) was added and the reaction stirred at room temperature for 18 hours before concentrating in vacuo. The residue was taken up in dichloromethane, washed with water and separated using a phase separation cartridge. The organic layer was dried over MgSO₄ and concentrated in vacuo. The residue was purified by preparative HPLC to afford the title compound.

LCMS Rt=3.21 min

MS m/z 382 [MH]+

### EXAMPLE 11

N-{6-Amino-5-[5-chloro-2-(trifluoromethoxy)phenyl]pyrazin-2-yl}-methyl-1H-pyrazole-5-carboxamide

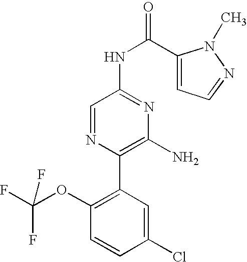

Oxalyl chloride (0.20 ml, 2.3 mmol) was added to a slurry of 1-methyl-1H-pyrazole-5-carboxylic acid (0.100 g, 0.793 mmol) in dichloromethane (10 ml). One drop dimethylformamide was added and the reaction left to stir at room temperature for 16 hours. The reaction was concentrated in vacuo and azeotroped with dichloromethane. The residue was dissolved in CH₃CN to give a 1M solution. 0.137 ml of the 1M solution of acid chloride (0.02 g, 0.137 mmol) in CH₃CN was added to a solution of the 3-[5-chloro-2-(trifluoromethoxy)phenyl]pyrazine-2,6-diamine (Preparation 3, 0.032 g, 0.106 mmol) and lutidine (0.02 ml, 0.18 mmol) in CH₃CN (2.5 ml). The reaction was warmed to room temperature and stirred for 18 hours before adding a further 0.05 ml of the acid chloride solution (0.05 mmol) and lutidine (0.01 ml, 0.09 mmol). After a further 3 hours at room temperature, the reaction was concentrated in vacuo. The residue was taken up in dichloromethane, washed with a saturated aqueous solution of NaHCO₃ and separated using a phase separation cartridge. The organic layer was dried over MgSO₄ and concentrated in vacuo. The residue was purified by preparative HPLC to afford the title compound.

LCMS Rt=3.36 min

MS m/z 413 [MH]+

### EXAMPLE 12

### N-{6-Amino-5-[2-(trifluoromethoxy)phenyl]pyrazin-2-yl}-3-(trifluoromethyl)isoxazole-4-carboxamide

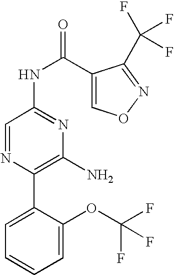

Oxalyl chloride (0.213 ml, 2.44 mmol) was added to a slurry of 3-trifluoromethyl-isoxazole-4-carboxylic acid (Preparation 22, 0.150 g, 0.828 mmol) in dichloromethane (5 ml). One drop dimethylformamide was added and the reaction left to stir at room temperature for 3 hours. The reaction was concentrated in vacuo and azeotroped with dichloromethane. The residue was dissolved in acetonitrile to give a 1M solution. 0.224 ml of the 1M solution of acid chloride (0.045 g, 0.224 mmol) in acetonitrile was added to a room temperature solution of the 3-[2-(trifluoromethoxy)-phenyl]pyrazine-2,6-diamine (Preparation 2, 0.055 g, 0.20 mmol) and lutidine (0.03 ml, 0.244 mmol) in acetonitrile (4 ml). The resultant solution was then stirred for 18 hours before adding a further 0.05 ml of the acid chloride solution (0.224 mmol) and lutidine (0.03 ml, 0.244 mmol). After a further 2 hours at room temperature, the reaction was concentrated in vacuo. The residue was taken up in ethyl acetate (70 ml) and hydrochloric acid (dilute aqueous solution, 30 ml) was added. The layers were separated and the organic layer was washed with a saturated aqueous solution of brine (30 ml) then NaHCO₃ (saturated aqueous solution, 30 ml). The organic layer was dried over anhydrous

MgSO₄ (s), filtered and evaporated in vacuo. The residue was purified by preparative HPLC to afford the title compound.

LCMS Rt=3.21 min

MS m/z 434 [MH]+

### EXAMPLE 13

N-{6-Amino-5-[5-fluoro-2-(trifluoromethoxy)phenyl]pyrazin-2-yl}-3-methylisoxazole-4-carboxamide

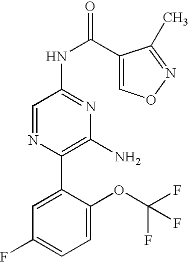

3-Methyl-isoxazole-4-carboxylic-acid-(6-amino-5-chloro-pyrazin-2-yl)-amide (Preparation 16, 0.035 g, 0.139 mmol) was combined with [2-(trifluoromethoxy)-5-fluorophenyl]boronic acid (Preparation 18, 0.050 g, 0.22 mmol), palladium tetrakistriphenylphosphine (0.016 g, 0.014 mmol) and cesium carbonate (0.045 g, 0.139 mmol) and suspended in a mixture of 1,4-dioxane (4 ml) and water (2 ml). The reaction was sealed and heated to 80° C. for 6 hours before cooling to room temperature. The reaction mixture was concentrated in vacuo then water (3 ml) and dichloromethane (3 ml) were added. The layers were separated and the organic layer was evaporated in vacuo. The residue was then purified by column chromatography on silica gel eluting with heptane:ethyl acetate 1:1 to afford the title compound as a white solid (6 mg, 11%).

LCMS Rt=1.44 min

MS m/z 398 [MH]+

### EXAMPLE 14

N-{6-Amino-5-[5-fluoro-2-(trifluoromethyl)phenyl]ovrazin-2-yl}-3-trifluoromethylisoxazole-4-carboxamide

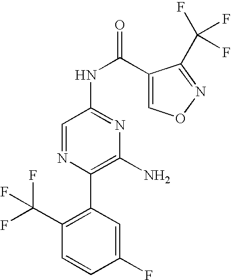

Oxalyl chloride (0.213 ml, 2.44 mmol) was added to a slurry of 3-tritluoromethyl-isoxazole-4-carboxylic acid (Preparation 22, 0.150 g, 0.828 mmol) in dichloromethane (5 ml). One drop dimethylformamide was added and the reaction left to stir at room temperature for 3 hours. The reaction was concentrated in vacuo and azeotroped with dichloromethane. The residue was dissolved in acetonitrile to give a 1M solution. 0.222 ml of the 1M solution of acid chloride (0.044 g, 0.222 mmol) in acetonitrile was added to a room temperature solution of the 3-[5-fluoro-2-(trifluoromethyl)phenyl]pyrazine-2,6-diamine (Preparation 5, 0.055 g, 0.20 mmol) and lutidine (0.03 ml, 0.242 mmol) in acetonitrile (4 ml). The resultant solution was then stirred for 18 hours before adding a further 0.05 ml of the acid chloride solution (0.222 mmol) and lutidine (0.03 ml, 0.242 mmol). After a further 2 hours at room temperature, the reaction was concentrated in vacuo. The residue was taken up in ethyl acetate (70 ml) and hydrochloric acid (dilute aqueous solution, 30 ml) was added. The layers were separated and the organic layer was washed with a saturated aqueous solution of brine (30 ml) then NaHCO₃ (saturated aqueous solution, 30 ml). The organic layer was dried over anhydrous MgSO₄ (5), filtered and evaporated in vacuo. The residue was purified by preparative HPLC to afford the title compound.

LCMS Rt=3.30 min

MS m/z 436 [MH]+

### EXAMPLE 15

N-{6-Amino-5-[5-fluoro-2-trifluoromethoxy)phenyl]pyrazin-2-yl}-1H-pyrazole-5-carboxamide

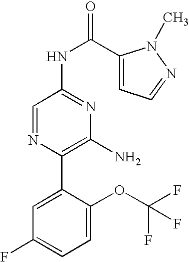

2-Methyl-2H-pyrazole-3-carboxylic acid-(6-amino-5-chloro-pyrazin-2-yl)-amide (Preparation 15, 0.035 g, 0.139 mmol) was combined with [2-(trifluoromethoxy)-5-fluorophenyl]boronic acid (Preparation 18, 0.050 g, 0.22 mmol), palladium tetrakistriphenylphosphine (0.016 g, 0.014 mmol) and cesium carbonate (0.045 g, 0.139 mmol) and suspended in a mixture of 1,4-dioxane (4 ml) and water (2 ml). The reaction was sealed and heated to 80° C. for 6 hours before cooling to room temperature. The reaction mixture was concentrated in vacuo then water (3 ml) and dichloromethane (3 ml) were added. The layers were separated and the organic layer was evaporated in vacuo. The residue was then purified by preparative HPLC.

LCMS Rt=3.12 min

MS m/z 397 [MH]+

### EXAMPLE 16

### N-{6-Amino-5-[5-ethoxy-2-(trifluoromethoxy)phenyl]pyrazin-2-yl}-1-methyl-1H-pyrazole-5-carboxamide

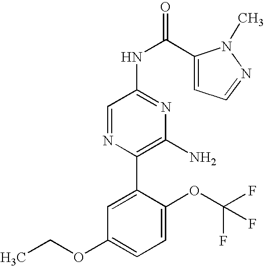

2-Methyl-2H-pyrazole-3-carboxylic acid-(6-amino-5-chloro-pyrazin-2-yl)-amide (Preparation 15, 0.030 g, 0.120 mmol) was combined with [2-(trifluoromethoxy)-5-ethoxy-phenyl]boronic acid (Preparation 24, 0.045 g, 0.178 mmol), palladium tetrakistriphenylphosphine (0.014 g, 0.013 mmol) and cesium carbonate (0.058 g, 0.178 mmol) and suspended in a mixture of 1,4-dioxane (3 ml) and water (1.5 ml). The reaction was sealed and heated under microwave conditions to 100° C. for 20 minutes before cooling to room temperature. The reaction mixture was concentrated in vacuo then saturated NaHCO₃ (aqueous solution, 3 ml) was added. The resultant solution was extracted with ethyl acetate (2×5 ml). The combined organic extracts were dried over anhydrous MgSO₄ (s), filtered and evaporated in vacuo. The residue was then purified by preparative HPLC.

LCMS Rt=3.15-3.20 min

MS m/z 423 [MH]+

### EXAMPLE 17

### N-(6-Amino-5-[5-ethoxy-2-{trifluoromethoxy)phenyl]pyrazin-2-yl}-3-methylisoxazole-4-carboxamide

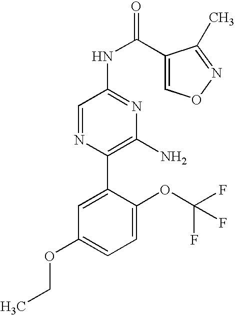

3-Methyl-isoxazole-4-carboxylic-acid-(6-amino-5-chloro-pyrazin-2-yl)-amide (Preparation 16, 0.040 g, 0.158 mmol) was combined with [2-(trifluoromethoxy)-5-ethoxy-phenyl]boronic acid (Preparation 24, 0.059 g, 0.237 mmol), palladium tetrakistriphenylphosphine (0.018 g, 0.016 mmol) and cesium carbonate (0.077 g, 0.237 mmol) and suspended in a mixture of 1,4-dioxane (3 ml) and water (1.5 ml). The reaction was sealed and heated to 50° C. for 5 hours before cooling to room temperature. The reaction mixture was concentrated in vacuo then saturated NaHCO₃ (aqueous solution, 3 ml) was added. The resultant solution was extracted with ethyl acetate (2×5 ml). The combined organic extracts were dried over anhydrous MgSO₄ (s), filtered and evaporated in vacuo. The residue was then purified by preparative HPLC.

LCMS Rt=3.13-3.20 min

MS m/z 424 [MH]+

### EXAMPLE 18

### N-{6-Amino-5-[5-fluoro-2-(trifluoromethoxy)phenyl]pyrazin-2-yl}-3-(trifluoromethyl)isoxazole-4-carboxamide

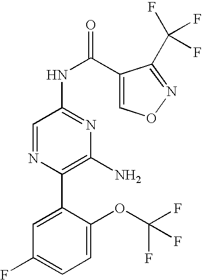

N-{6-Amino-5-[5-fluoro-2-(trifluoromethoxy)phenyl]pyrazin-2-yl}-3-(trifluoromethyl)isoxazole-4-carboxamide may be prepared by processes analogous to those described above.

The following Preparations illustrate the preparation of certain intermediates used to prepare the above Examples.

### Preparation 1

### Methyl 3,5-diamino-6-[2-(trifluoromethoxy)phenyl]pyrazine-2-carboxylate

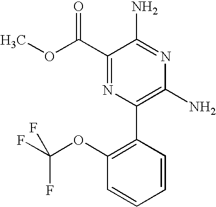

### Method A

Methyl 3,5-diamino-6-chloropyrazine-2-carboxylate (1.62 g, 8.0 mmol) was combined with 2-(trifluoromethyl)phenylboronic acid (3.29 g, 16.0 mmol), palladium tetrakistriphenylphosphine (0.924 g, 0.80 mmol) and cesium carbonate (2.61 g, 8.0 mmol) and suspended in a mixture of 1,4-dioxane (30 ml) and water (15 ml). The reaction was sealed and heated to 75° C. for 4 hours before cooling to room temperature. Water (150 ml) was added and the 1,4-dioxane removed in vacuo. The precipitate formed was collected by filtration and dried. The brown solid was triturated with dichloromethane:methanol, then purified by silica gel column chromatography eluting with 95:5 dichloromethane:methanol to afford the title compound as a yellow solid (1.66 g, 63% yield).

¹HNMR (d₆-DMSO): 3.7 (s, 3H), 6.59 (br s, 2H), 7.10 (br s, 2H), 7.41-7.64 (m, 4H)

LCMS Rt=8.64 min

MS m/z 329 [MH]+

### Preparation 2

### 3-[2-(Trifluoromethoxy)phenyl]pyrazine-2,6-diamine

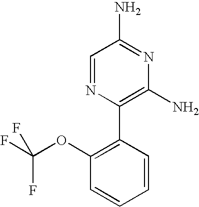

### Method B

To a suspension of methyl 3,5-diamino-6-{2-(trifluoromethoxy)phenyl}pyrazine-2-carboxylate (Preparation 1, 0.35 g, 1.07 mmol) in methanol (15 ml) and water (5 ml) was added lithium hydroxide (0.134 g, 3.18 mmol). The reaction was stirred at 90° C. for 1 hour before concentrating in vacuo to afford a brown gum. The residue (0.335 g, 1.07 mmol) was slurried in 1,4-dioxane (20 ml) and to this was added 2N HCl (12 ml). The reaction was heated to 100° C. for 1.5 hours before cooling to room temperature and concentrating in vacuo. The residue was basified with saturated aqueous K₂CO₃ and extracted with ethyl acetate. The organic layer was then dried over MgSO₄ and concentrated in vacuo to afford the title compound as an orange gum (0.160 g, 55% yield)

¹HNMR (d₆-DMSO): 5.2 (br s, 2H), 5.9 (br s, 2H), 7.2 (m, 1H), 7.4-7.6 (m, 4H)

LCMS Rt=0.64 min MS m/z 271 [MH]+

The following Preparations of the general formula:

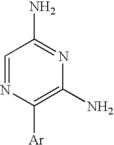

were prepared by a 2-step method analogous to Method A followed by Method B. Unless otherwise noted, preparation details are as described for the method referred to.

| Preparation | | | |
|---|---|---|---|
| No. | | | |
| Name | Ar | Data | Preparation Information |
| 3 | 5-chloro-2- | LCMS Rt = 1.53 min | Method A in a small, |
| 3-[5-Chloro-2- | (trifluoromethoxy)phenyl | MS m/z 305 [MH]+ | sealed, reaction vial |
| (trifluoromethoxy)phenyl]pyrazine- | | ¹HNMR (d₆- | (Reacti-vial ™), using 4 |
| 2,6- | | DMSO): 5.52 (br s, | equivalents of a mixture of |
| diamine | | 2H), 6.04 (br s, | 5-chloro-2- |
| | | 2H), 7.17 (s, 1H), | (trifluoromethoxy)phenylboronic |
| | | 7.45 (d, 1H), | acid and 2-chloro-5- |
| | | 7.46 (s, 1H), 7.52 (d, | (trifluoromethoxy)phenylboronic |
| | | 1H). | acid (Preparation 6), |
| | | HSQC performed | 1.1 equivalents cesium |
| | | to confirm | carbonate and 0.079 |
| | | structure. | equivalents palladium |
| | | | tetrakis(triphenylphosphine). |
| | | | Catalyst added at 75° C. |
| | | | Regioisomers separated |
| | | | by silica gel column |
| | | | chromatography eluting |
| | | | with 30:70 to 60:40 ethyl |
| | | | acetate:heptane. |
| | | | Method B, using 3 |
| | | | equivalents LiOH, at 90° C. |
| | | | for 4 hours, followed by 2M |
| | | | HCl at 100° C. for 4 hours. |
| 4 | 2- | LCMS Rt = 1.64 min | Method A, using 2- |
| 3-[2- | (trifluoromethyl)phenyl | MS m/z 255 [MH]+ | (trifluoromethyl)phenylboronic |
| (Trifluoromethyl)phenyl]pyrazine- | | ¹HNMR (d₆- | acid. |
| 2,6-diamine | | DMSO): 5.5 (br, | Method B, using 3 |
| | | 2H), 6.1 (br, 2H), | equivalents LiOH, at 90° C. |
| | | 7.18 (s, 1H), | for 3 hours, followed by 2M |
| | | 7.4 (d, 1H), 7.5-7.7 (m, | HCl at 100° C. for 4 hours. |
| | | 2H), 7.8 (d, 1H). | |
| 5 | 5-fluoro-2- | LCMS Rt = 2.41 min | Method A, using 1.6 |
| 3-[5-Fluoro-2- | (trifluoromethyl)phenyl | MS m/z | equivalents 5-fluoro-2- |
| (trifluoromethyl)phenyl]pyrazine- | | 273 [MH]+ | (trifluoromethyl)phenylboronic |
| 2,6-diamine | | ¹HNMR (d₆- | acid and 0.05 |
| | | DMSO): 5.4 (br, | equivalents palladium |
| | | 2H), 5.95 (br, 2H), | tetrakis(triphenylphosphine). |
| | | 7.1 (s, 1H), 7.2 (m, | Reaction stirred at 80° C. |
| | | 1H), 7.4 (m, 1H), | for 18 hours. Further 0.01 |
| | | 7.8 (m, 1H). | equivalents catalyst and |
| | | | 0.16 equivalents boronic |
| | | | acid added and stirred at |
| | | | 80° C. for a further 18 |
| | | | hours. |
| | | | Method B, using 3 |
| | | | equivalents LiOH at 90° C. |
| | | | for 2 hours, followed by 2M |
| | | | HCl at 100° C. for 2 hours. |

### Preparation 6

### [5-Chloro-2-(trifluoromethoxy)phenyl]boronic Acid

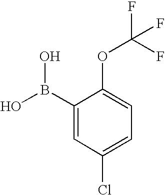

Boron trifluoride etherate (0.415 ml, 3.56 mmol) and trimethyl borate (0.794 ml, 7.12 mmol) were stirred in diethyl ether (10 ml) for 10 minutes to form dimethoxyfluoroborane in situ.

To a solution of the 4-chloro(trifluoromethoxy)benzene (2.0 g, 10.18 mmol) in dry tetrahydrofuran (THF, 30 ml) at −78° C., was added ethylenediaminetetraacetic acid (EDTA, 1.24 g, 10.7 mmol) followed by a 1.3 M solution of sec-butyllithium in cyclohexane (7.63 ml, 10.7 mmol) and the reaction stirred for 2 hours under nitrogen.

To this reaction mixture at −78° C., was then added dropwise the preformed dimethoxyfluoroborane mixture. The reaction was stirred at −78° C. for 30 minutes, warmed to room temperature for 30 minutes, and then quenched with water (10 ml). The reaction mixture was extracted with diethyl ether (4×50 ml). The combined organic extracts were dried over MgSO₄ and concentrated in vacuo. The residue was dissolved in diethyl ether (10 ml) and washed with an aqueous solution of 10% NaOH (50 ml). The aqueous layer was acidified and extracted with ethyl acetate (3×40 ml). The combined ethyl acetate extracts were dried over MgSO₄ and concentrated in vacuo to afford a mixture of the title compound and its corresponding regioisomer as a white solid (0.862 g). Regioisomers were not separated.

LCMS Rt=1.42 min

MS m/z 239 [M]

### Preparation 7

### 3-(Methoxymethyl)isoxazole-4-carboxylic Acid and 3-(Methoxymethyl)isoxazole-5-carboxylic Acid

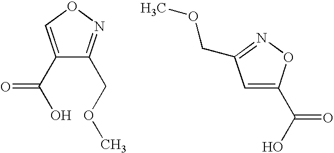

To a solution of 3-(methoxymethyl)isoxazole-4-carboxylic acid methyl ester and 3-(methoxymethyl)isoxazole-5-carboxylic acid methyl ester prepared as a mixture of isoxazole regioisomers (Preparation 8, 2.0 g, 2.3 mmol) in 1,4-dioxane (20 ml) was added an aqueous solution of sodium hydroxide (0.5 g, 12.5 mmol in 5 ml water) and the reaction stirred vigorously at room temperature for 1 hour. The reaction was concentrated in vacuo, and the residue partitioned between t-butylmethyl ether (80 ml) and water (30 ml). The aqueous layer was separated and acidified with concentrated hydrochloric acid before extracting with t-butylmethyl ether. The organic layer was then dried over Na₂SO₄ and concentrated in vacuo to afford a 1:5 mixture of regioisomers. The solid was dissolved in warm t-butylmethyl ether (1-0 ml) and heptane (10 ml) added. The re-cyrstallisation liquors were concentrated in vacuo to afford a mixture of isoxazole regioisomers, enriched with 3-(methoxymethyl)isoxazole-5-carboxylic acid as a 1:3 ratio 0.3 g, 16% yield)

¹HNMR (CDCl₃): 3.36 (s, 2.25H), 3.45 (s, 0.75H), 4.55 (s, 1.5H), 4.75 (s, 0.5H), 7.04 (s, 0.75H), 8.96 (s, 0.25H)

### Preparation 8

### 3-(Methoxymethyl)isoxazole-4-carboxylic acid methyl ester and 3-(Methoxymethyl)isoxazole-5-carboxylic Acid Methyl Ester

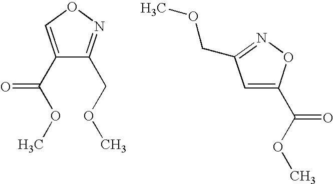

To a cooled solution of N-hydroxy-2-methoxyethanimidoyl chloride (Preparation 9, 2.0 g, 16.19 mmol) and methyl propiolate (3 ml, 33.0 mmol) in toluene (20 ml) was added dropwise diisopropylethylamine (3 ml, 17.0 mmol). The reaction was stirred at room temperature for 1 hour. t-Butylmethyl ether (50 ml) and water (50 ml) were added to the mixture and the pH of the aqueous layer adjusted to pH 1-2 with 2M hydrochloric acid. The organic layer was dried over Na₂SO₄ and concentrated in vacuo to afford the title compounds as an inseparable mixture of isoxazole regioisomers (2.0 g, 72% yield).

¹HNMR (CDCl₃): 3.41 (s, 2.55H), 3.48 (s, 0.45H), 3.89 (s, 2.55H), 3.98 (s, 0.45H), 4.59 (s, 1.7H), 4.79 (s, 0.3H), 7.06 (s, 0.84H), 8.90 (s, 0.15H)

### Preparation 9

### N-Hydroxy-2-methoxyethanimidoyl Chloride

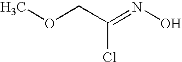

To a cooled solution of methoxyacetaldehyde oxime (Preparation 10, 1.5 g, 16.84 mmol) in dimethylformamide (7 ml) was added N-chlorosuccinimide (2.3 g, 17.22 mmol) and the reaction stirred at room temperature for 1 hour. The reaction was concentrated in vacuo and the residue partitioned between t-butylmethyl ether (100 ml) and water (50 ml). The organic layer was dried over Na₂SO₄ and concentrated in vacuo, to afford the title compound as a colourless oil (2.0 g, 96% yield)

¹HNMR (CDCl₃): 3.4 (s, 3H), 4.2 (s, 2H), 8.61 (br s, 1H)

### Preparation 10

### Methoxyacetaldehyde Oxime

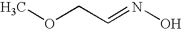

To a solution of methoxyacetaldehyde dimethylacetal (5 g, 41.63 mmol) in methanol (20 ml) was added a solution of hydroxylamine hydrochloride (2.9 g, 41.73 mmol) in water (10 ml). The reaction was stirred at room temperature for 18 hours. To the reaction was then added an aqueous solution of sodium hydroxide (1.67 g, 41.6 mmol in 10 ml water) and stirred for 3 hours at room temperature. The methanol was removed in vacuo and the mixture acidified with concentrated hydrochloric acid to pH 5-6, before extracting with t-butylmethyl ether, drying over Na₂SO₄ and concentrating in vacuo to afford the title compound as a 1.5:1 mixture of E/Z isomers (2.64 g, 71% yield).

¹HNMR (CDCl₃): 3.4 (m, 3H), 4.05 (d, 1.2H), 4.3 (d, 0.8H), 6.9 (t, 0.4H), 7.5 (t, 0.6H), 8.55 (br s, 0.6H), 8.85 (br s, 0.4H)

### Preparation 11

### 5-(Methoxymethyl)isoxazole-4-carboxylic Acid

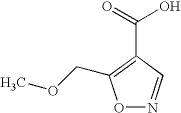

Methyl 5-(methoxymethyl)isoxazole-4-carboxylate (Preparation 12, 1.8 g, 11 mmol) was stirred in a 1:1:1 mixture of concentrated hydrochloric acid (2 ml), acetic acid (2 ml) and water (2 ml) at reflux for 6 hours. Acetone (6 ml) was added and the mixture concentrated in vacuo. The solid residue was triturated with ethyl acetate and the filtrate concentrated in vacuo to afford the title compound as an off white solid (1.4 g, 85% yield).

LCMS Rt=0.86 min

MS m/z 157 [MH]+

¹HNMR (CDCl₃): 3.52 (s, 3H), 4.91 (s, 2H), 8.61 (s, 1H)

### Preparation 12

### Methyl 5-(methoxymethyl)isoxazole-4-carboxylate

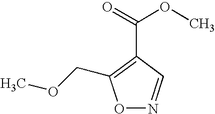

To a solution of methyl 2-[(dimethylamino)methylene]-4-methoxy-3-oxobutanoate (Preparation 13, 5.2 g, 26 mmol) in methanol (55 ml) was added hydroxylamine hydrochloride (1.8 g, 25.8 mmol) and the reaction stirred at reflux for 7 hours. The reaction was concentrated in vacuo. The solid residue was purified by trituration with ethyl acetate to afford the title compound as a solid (3.6 g, 18% yield).

¹HNMR (CDCl₃): 3.48 (s, 3H), 3.89 (s, 3H), 4.87 (s, 2H), 8.53 (s, 1H)

LCMS Rt=1.06 min

MS m/z 172 [MH]+

### Preparation 13

### Meth 12 dimeth-3-oxobutanoate

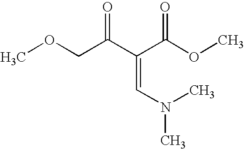

Methyl-4-methoxyacetoacetate ml, 70 mmol) was added to dimethylformamide dimethylacetal (18.8 ml, 139 mmol) and the reaction stirred at 90° C. for 2 hours before cooling to room temperature and stirring for 18 hours. The reaction was concentrated in vacuo and purified by silica gel column chromatography, eluting with 70:30 to 100:0 ethyl acetate:heptane to afford the title compound as an oil (7.68 g, 50% yield).

¹HNMR (CDCl₃): 2.87 (br s, 3H), 3.295/br s, 3H), 3.39 (s, 3H), 3.72 (s, 3H), 4.37 (s, 2H), 7.74 (s, 1H)

### Preparation 14

### 3-Chloro-pyrazine-2,6-diamine

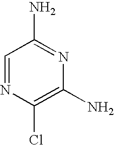

Lithium hydroxide (12.4 g, 0.30 mol) was added to a stirred suspension of 3,5-diamino-6-chloro-pyrazine-2-carboxylic acid methyl ester (20 g, 99 mmol) in methanol (300 ml) and water (120 ml) and the reaction heated at 90° C. for 1.5 hours before allowing to cool to room temperature. The reaction was concentrated in vacuo to afford a yellow slurry and this was suspended in 1,4-dioxane (350 ml) and 2M aqueous HCl solution (200 ml,) was added. The mixture was heated at 100° C. for 2 hours and then allowed to cool before removing the 1,4-dioxane in vacuo. The resulting aqueous solution was taken to pH 8 using sodium carbonate (saturated aqueous) and extracted into ethyl acetate (3×300 ml). The combined organic layers were washed with brine (300 ml), dried (Na₂SO₄) and concentrated in vacuo to afford a yellow solid (11.7 g, 82%).

¹HNMR (d₆-DMSO):

### Preparation 15

### 2-Methyl-2H-pyrazole-3-carboxylic Acid-(6-amino-5-chloro-pyrazin-2-yl)-amide

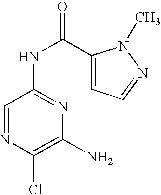

Oxalyl chloride (0.288 ml, 3.315 mmol) was added to a slurry of 2-methyl-2H-pyrazole-3-carboxylic acid (279 mg, 2.21 mmol) in dichloromethane (3 ml). One drop of N,N-dimethylformamide was added and the reaction left to stir at room temperature for 4 hours. The reaction was concentrated in vacuo and azeotroped with dichloromethane. The residue was dissolved in anhydrous pyridine (2 ml) and was added to a solution of the 3-chloro-pyrazine-2,6-diamine (Preparation 14, 160 mg, 1.107 mmol) in anhydrous pyridine (3 ml). The reaction was warmed to 60° C. and stirred for 3 hours. The reaction mixture was cooled to room temperature then concentrated in vacuo. The residue was purified by column chromatography on silica gel eluting with heptane:ethyl acetate 1:1 to afford the title compound as a yellow solid (100 mg, 36%).

¹HNMR (d₆-DMSO): 4.05 (s, 3H), 6.60 (br s, 2H), 7.20 (d, 1H), 7.50 (d, 1H), 8.30 (s, 1H), 10.60 (br s, 1H).

LCMS Rt=2.21 min

MS m/z 253 [MH]+

### Preparation 16

### 3-Methyl-isoxazole-4-carboxylic acid-(6-amino-5-chloro-pyrazin-2-yl)-amide

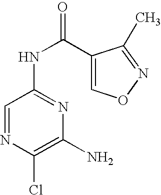

Oxalyl chloride (0.060 ml, 0.691 mmol) was added to a slurry of 3-methyl-isoxazole-4-carboxylic acid (60 mg, 0.476 mmol) in dichloromethane (3 ml). One drop of N,N-dimethylformamide was added and the reaction left to stir at room temperature for 4 hours. The reaction was concentrated in vacuo and azeotroped with dichloromethane. The residue was dissolved in anhydrous pyridine (1 ml) and was added to a solution of the 3-chloro-pyrazine-2,6-diamine (Preparation 14, 35 mg, 0.238 mmol) in anhydrous pyridine (2 ml). The reaction was warmed to 50° C. and stirred for 3 hours. The reaction mixture was cooled to room temperature then concentrated in vacuo. The residue was purified by column chromatography on silica gel eluting with heptane:ethyl acetate 1:1 to afford the title compound as a white solid (30 mg, 50%).

¹HNMR (d₆-DMSO): 2.40 (s, 3H), 6.60 (br s, 2H), 8.35 (s, 1H), 9.60 (s, 1H), 10.65 (br s, 1H).

LCMS Rt=2.34-2.37 min

MS m/z 254 [MH]+

### Preparation 17

### [2-Trifluoromethoxy)-5-fluoro-1-bromo]benzene

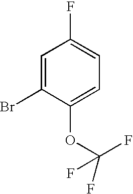

To a stirred solution of 3-bromo-4-trifluoromethoxyaniline (30 g, 0.12 mol) in hydrochloric acid (6N aqueous solution) (300 ml) was added drop-wise a solution of sodium nitrite (9.7 g, 0.14 mol) in water (30 ml) at 0° C. The resulting mixture was stirred at 0-5° C. for 1 hour until the reaction system became clear. Tetrafluoroboronic acid (40% aqueous solution) (90 ml) was then added drop-wise over 15 minutes. The resulting mixture was again stirred at 0-5° C. for 1 hour then filtered. The filter cake was washed with cold water (100 ml) and diethyl ether (100 ml), then dried in vacuo to give the hydrazinium tetrafluoroborate salt as a white solid (35 g, 84%). This solid (8.5 g, 0.024 mol) was then slowly heated to 140° C. and maintained at this temperature for 1 hour under an atmosphere of nitrogen. The reaction mixture was cooled to room temperature and distilled under reduced pressure to afford the title compound as a colourless oil (4.86 g, 78%).

¹HNMR (CDCl₃): 7.02-7.09 (m, 1H), 7.26-7.29 (m, 1H), 7.33-7.38 (m, 1H).

LCMS (30 min) Rt=6.9 mins;

MS m/z 258 [MH]+

### Preparation 18

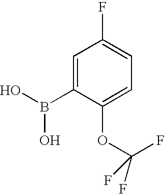

A solution of isopropylmagnesium bromide (2M solution in tetrahydrofuran) (83 ml, 0.166 mol) was added drop-wise to a stirred solution of 2-(trifluoromethoxy)-5-fluoro-1-bromobenzene (Preparation 17, 27.6 g, 0.107 mol) in anhydrous tetrahydrofuran (125 ml) at −10° C. under an atmosphere of nitrogen. The resulting mixture was stirred at room temperature for 2 hours. Triisopropyl borate (26.1 g, 0.139 mol) was then added drop-wise at −10° C. and the resulting mixture was stirred at room temperature for 16 hours. Hydrochloric acid (1N aqueous solution) (100 ml) was added drop-wise at 0° C. and the mixture stirred at room temperature for 30 minutes. Ethyl acetate (150 ml) was added and the layers were separated, the aqueous layer was further extracted with ethyl acetate (2×150 ml). The organic extracts were combined and concentrated in vacuo. The residue was dissolved in potassium hydroxide (10% aqueous solution) (50 ml) and extracted with diethyl ether (2×150 ml). The separated aqueous layer was acidified to pH-0.4 by addition of hydrochloric acid (1N aqueous solution) (100 ml) and extracted with ethyl acetate (3×150 ml). The combined organic extracts were dried over anhydrous Na₂SO₄, filtered and evaporated in vacuo to give an off white solid. Purification by preparative HPLC gave the title compound as an off white solid (5.82 g, 24%).

¹HNMR (d₆-DMSO): 7.23-7.32 (m, 3H), 7.53-7.55 (m, 1H), 8.36 (br s, 1H).

MS m/z 223 [MH]−

### Preparation 19

### Trifluoro-acetaldehyde Oxime

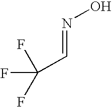

To a solution of trifluoroacetaldehydemethyl hemiacetal (10 g, 77 mmol) and hydroxylamine hydrochloride (5.50 g, 79 mmol) in methanol (15 ml) and water (35 ml) at 0° C. was slowly added sodium hydroxide (50% aqueous solution) (18 ml). The reaction mixture was then allowed to warm to room temperature with stirring over 1′6 hours. Heptane 50 ml) was added and the layers separated. The aqueous layer was then acidified by addition of hydrochloric acid (6M aqueous solution) (30 ml) then extracted with diethyl ether (2×100 ml). The organic extracts were combined and dried over anhydrous Na₂SO₄(s), filtered and evaporated at atmospheric pressure to afford the crude title compound as a 1:2 etherate, as a colourless oil (16.77 g, containing 7.5 g of oxime, 86.3%). Material was taken on without further purification.

¹HNMR (CDCl₃): 7.45-7.50 (m, 1H), 9.58 (s, 1H).

### Preparation 20

### N-Hydroxy-2-trifluoromethylethanimidoyl Bromide

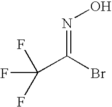

To an ice cooled solution of trifluoro-acetaldehyde oxime (Preparation 19, 16.77 g of a 2:1 etherate containing 7.5 g, 66.3 mmol of the oxime) in anhydrous N,N-dimethylformamide (10 ml) was added a solution of N-bromosuccinimide (12 g, 67 mmol) in anhydrous N,N-dimethylformamide (20 ml), drop-wise, over a period of 45 minutes. The reaction mixture was then warmed to room temperature with stirring over 4 hours. Diethyl ether (150 ml) and water (100 ml) were added and the layers separated. The organic layer was dried over anhydrous Na₂SO₄ (s), filtered and evaporated at atmospheric pressure to afford the crude title compound as a 1:1.5 etherate, as a yellow oil (17.4 g, containing 12.0 g of oxime, 94%). Material was taken on without further purification.

¹HNMR (CDCl₃): 8.02 (s, 1H).

### Preparation 21

### 3-Trifluoromethyl-isoxazole-4-carboxylic Acid Ethyl Ester

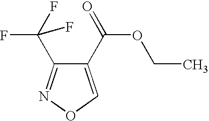

To a solution of dimethylamino acrylate g, 35 mmol) in toluene (50 ml) was added bromo-oxime (Preparation 20, 6.0 g plus ether, 31 mmol), drop-wise, and the resultant solution was stirred for three hours at room temperature. The reaction mixture was evaporated to dryness, then t-butylmethyl ether (60 ml) and water (20 ml) were added. The layers were separated and the organic layer was washed with dilute hydrochloric acid (20 ml), then water (20 ml) and brine (10 ml). The organic fraction was then dried over anhydrous Na₂SO₄ (s), filtered and evaporated in vacuo to afford the title compound as an orange/brown oil (4.65 g, 72%). Material was taken on with no further purification.

¹HNMR (CDCl₃): 1.35 (t, 3H), 4.36 (q, 2H), 9.03 (s, 1H).

### Preparation 22

### 3-Trifluoromethyl-oxazole-4-carboxylic Acid

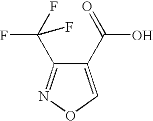

3-Trifluoromethyl-isoxazole-4-carboxylic acid ethyl ester (Preparation 21, 1.00 g, 4.78 mmol), glacial acetic acid (4 ml), concentrated hydrochloric acid (2 ml, 20 mmol) and water (2 ml, 200 mmol) were heated together with stirring at 70° C. for 2 hours. Solvents were removed by evaporation in vacuo and the residue was left to stand at room temperature for 16 hours. Water (40 ml) and t-butylmethyl ether (80 ml) was added and the layers separated. The organic layer was washed with dilute hydrochloric acid (20 ml), then dried over anhydrous Na₂SO₄ (s), filtered and evaporated in vacuo to afford the title compound as a brown gum (70 mg, 8%). Material was taken on with no further purification.

### Preparation 23

### 2-Bromo-4-ethoxy-1-trifluoromethoxy-benzene

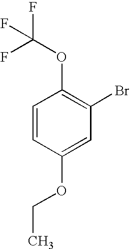

To a solution of the 3-bromo-4-trifluoromethoxyphenol (1.0 g, 2.48 mmol) in acetone (30 ml) was added ethyl iodide (0.795 ml, 9.94 mmol) followed by potassium carbonate (1.37 g, 9.94 mmol) and resulting solution was heated to reflux for 12 hrs. The reaction mixture was cooled then filtered and concentrated in vacuo. Dichloromethane (20 ml) and water (20 ml) were added and the solution was filtered through a phase separation cartridge. The organic layer was collected, and evaporated in vacuo to afford crude title compound as a colourless oil (884 mg, 80%). Material was taken on without further purification.

¹H-NMR (d₆-DMSO) 1H, 1.25 (t, 3H), 4.05 (q, 2H), 7.00 (dd, 1H), 7.35 (d, 1H), 7.40 (dd, 1H).

LCMS (2 min) Rt=1.82 min

MS m/z 286 [MH]+

### Preparation 24

### [2-(Trifluoromethoxy)-5-ethoxy-phenyl]boronic Acid

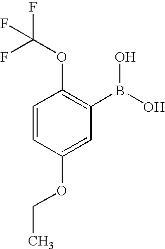

To a stirred solution of the 2-bromo-4-ethoxy-1-trifluoromethoxy-benzene (Preparation 23, 884 mg, 3.10 mmol) in anhydrous tetrahydrofuran (10 ml) was added n butyl lithium (2M solution in cyclohexanes, 2.33 ml, 4.65 mmol) while maintaining the temperature below −70° C. under an atmosphere of nitrogen. The solution was stirred at this temperature for 1 hour then tri-isopropylborate (875 mg, 4.65 mmol) was added, and the reaction maintained at −70° C. for a further 2 hours. The reaction mixture was then quenched by the addition of ammonium chloride (aqueous solution) (5 ml), followed by acidification with hydrochloric acid (2N aqueous solution) (10 ml). The layers were separated and the organic layer was dried over anhydrous MgSO₄ (s), filtered and evaporated in vacuo to afford crude title compound as a white solid (552 mg, 71%). Material was taken on without further purification.

The ability of the pyrazine derivatives of the formula (I) to inhibit the Na_{V1.8} channel may be measured using the assay described below.

### VIPR Assay for Nav1.8 Compounds

This screen is used to determine the effects of compounds on tetrodotoxin-resistant (TTX-R) sodium channels in Human Nav1.8 (HEK293) expressing cell line, utilising the technology of Aurora's fluorescent Voltage/Ion Probe Reader (VIPR). This experiment is based on FRET (Fluorescence Resonance Energy Transfer) and uses two fluorescent molecules. The first molecule, Oxonol (DiSBAC₂(3)), is a highly fluorescent, negatively charged, hydrophobic ion that “senses” the trans-membrane electrical potential. In response to changes in membrane potential, it can rapidly redistribute between two binding sites on opposite sides of the plasma membrane. The voltage dependent redistribution is transduced into a ratiometric fluorescent readout via a second fluorescent molecule (Coumarin (CC2-DMPE)) that binds specifically to one face of the plasma membrane and functions as a FRET partner to the mobile voltage-sensing ion. To enable the assay to work, the channels have to be pharmacologically held in the open state. This is achieved by treating the cells with either deltamethrin (for Na_{V1.8}) or veratridine (for the SHSY-5Y assay for TTX-S channels).

### Cell Maintenance:

Human Nav1.8 cells are grown in T225 flasks, in a 5% CO2 humidified incubator to about 70% confluence. Media composition consists of DMEM/F-12, 10% FCS and 300 μg/ml Geneticine. They are split using cell dissociation fluid 1:5 to 1:20, depending on scheduling needs, and grown for 3-4 days before the next split.

### Protocol:

### Day One:

Plate-out HEK-Nav1.8 cells (100 μl per well) into poly-D-lysine coated plates prior to experimentation as follows:—24 hours @ 3.5×10⁴ cells/well (3.5×10⁵ cells/ml) or using the technology of Select.

### Day Two: VIPR Assay:

1. Equilibrate buffers at room temperature for 2 hours or at 37° C. for 30 minutes prior to experimentation.
2. Prepare Coumarin dye (see below) and store in dark. Prime the plate washer with Na+ Free buffer and wash cells twice, Note: Plate washer deposits ˜30 μl residual buffer per well. Add 1004 Coumarin (CC2-DMPE) solution (see below) to cells and incubate for 45 minutes at room temperature avoiding bright light.
3. Prepare Oxonol (DiSBAC₂(3)) dye (see below):
4. Aspirate off Coumarin solution from the cells by washing in Na+ Free buffer.
5. Add 30 μl compound then add 30 μl Oxonol solution to the cells and incubate for 45 minutes at room temperature in the dark (total well volume ˜90 μl).
6. Once the incubation is complete, the cells are ready to be assayed using the VIPR for sodium addback membrane potential.

The data was analyzed and reported as normalised ratios of intensities measured in the 460 nm and 580 nm channels. The process of calculating these ratios was performed as follows. An additional plate contained control solution with the same DisBAC2(3) concentrations as used in the cell plates, however no cells were included in the background plate. Intensity values at each wavelength were averaged for sample points 5-7 (initial) and 44-49 (final). These averages were subtracted from intensity values averaged over the same time periods in all assay wells. The initial ratio obtained from samples 3-8 (Ri) and the final ratio obtained from samples 45-50 (Rf) are defined as:

Ri=(Intensity 460 nm, samples 3-5—background 460 nm, samples 3-5)

(Intensity 580 nm, samples 3-5—background 580 nm, samples 3-5)

Rf=(Intensity 460 nm, samples 25-30—background 460 nm, samples 25-30)

(Intensity 580 nm, samples 25-30—background 580 nm, samples 25-30)

Final data are normalised to the starting ratio of each well and reported as Rf/Ri. This analysis is performed using a computerised specific programme designed for VIPR generated data.
Rf/Ri ratio values are plotted using Excel Labstats (curve fit) or analysed via ECADA to determine an IC50 value for each compound.
Na+—Addback Buffer pH 7.4 (adjust with 5M NaOH)—10× stock

| | | weight/ | 10X | |
|---|---|---|---|---|
| Component: | Mwt/Concⁿ: | volume | Conc. (mM) | 1X Conc. (mM): |
| NaCl | 58.44 | 93.5 g | 1600 | 160 |
| KCL | 74.55 | 3.35 g | 45.0 | 4.5 |
| CaCl₂ | 1M solution | 20 ml | 20.0 | 2 |
| MgCl₂ | 203.31 | 2.03 g | 10.0 | 1 |
| Hepes | 238.3 | 23.83 g | 100 | 10 |
| dH₂O | 1 L | | | |

Na+—Free Buffer pH 7.4 (adjust with 5M KOH)—10× stock

| | | weight/ | 10X | 1X |
|---|---|---|---|---|
| Component: | Mwt/Concⁿ: | volume | Conc.(mM) | Conc (mM): |
| Choline chloride | 139.6 | 223.36 g | 1600 | 160 |
| CaCl₂ | 1M solution | 1 ml | 1.0 | 0.1 |
| MgCl₂ | 203.31 | 2.03 g | 10.0 | 1.0 |
| Hepes | 238.3 | 23.83 g | 100 | 10 |
| dH₂O | 1 L | | | |

1×Na+ Free Buffer: −400 ml 10×+3600 ml dH2O
2×Na+ Free Buffer: −100 ml 10×+400 ml dH2O
1×Na+ Addback Buffer: −50 ml 10×Na+Addback+450 ml dH2O
Coumarin (CC2-DMPE): For 2 plates: —
First mix 220 μl Coumarin (1 mM)+120 μl Pluronic (20%) in a tube+22 ml 1×Na+−Free Buffer, gently vortex.

| | Solution Concⁿ: | Final Assay Concⁿ |
|---|---|---|
| Coumarin (1 mM) | 10 μM | 10 μM |

Oxonol (DiSBAC₂(3)): For 2 plates:—
48 μl Oxonol (5 mM)+120 ul Tartrazine (200 mM) Vortex

### 8.0 ml 2×Na+−Free Buffer Vortex

### 1.6 μl Deltamethrin (5 mM) Vortex

| | Solution | |
|---|---|---|
| | Concⁿ: | Final Assay Concⁿ |
| Oxonol (5 mM) | 30 μM | 10 μM |
| Deltamethrin (5 mM) | 1 μM | 330 nM |
| Tartrazine (200 mM) | 3 mM | 1.0 mM |

### TTX-S Assay

The TTX-S assay is performed in the SHSY-5Y cell line which constitutively express a number of tetrodotoxin-sensitive voltage-gated sodium channels including Na_{V1.2}, Na_{V1.3} and Na_{V1.7}. The procedure detailed above for the Na_{V1.8} assay was followed with the exception that veratridine was substituted for deltamethrin in the assay as an opener of the sodium channels, at a final assay concentration of 50 μM.

### Nav_{1.5} Assay

The Nav_{1.5} assay is performed in HEK293 cells expressing Human Nav_{1.5} in the same way as the Nav_{1.8} assay described above.

Compounds of the Examples were tested in the assays described above.

| | Na_{V1.8} | Na_{V1.5} | TTX-S |
|---|---|---|---|
| Example | IC50 | IC50 | IC50 |
| No. | (μM) | (μM) | (μM) |
| 1 | 3.6 | >32 | 30 |
| 2 | 8.70 | — | >32 |
| 3 | 4.3 | >32 | 16 |
| 4 | 27 | >32 | >32 |
| 5 | 19 | >32 | 29 |
| 6 | 20 | >32 | 19 |
| 7 | 4.6 | 21 | 17 |
| 8 | 11 | >32 | — |
| 9 | 20 | >32 | 30 |
| 10 | 4.7 | >32 | >32 |
| 11 | 12 | >32 | >32 |
| 12 | 2.8 | >32 | 21 |
| 13 | 3.8 | >32 | >32 |
| 14 | 2.5 | >32 | 31 |
| 15 | 16 | >32 | >32 |
| 16 | 19 | >32 | — |
| 17 | 1.9 | >32 | — |
| 18 | — | — | — |

Where replicate experiments were conducted resulting in multiple sets of data for a test compound, the data presented represent the average value from all replicate experiments.

1. A compound of the formula (I): 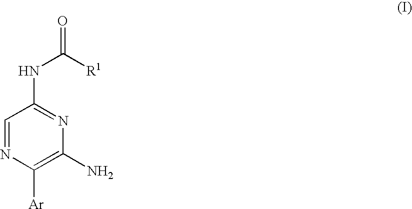
or a pharmaceutically acceptable salt or solvate thereof;
wherein Ar is 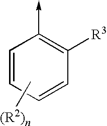
wherein→indicates the point of attachment to the pyrazine ring;
each R² is independently selected from (C₁-C₄)alkyl, (C₁-C₄)alkoxy, halo(C₁-C₄)alkyl, halo(C₁-C₄)alkoxy, cyano and halo;
n is 0 to 4;
R³ is CF₃ or OCF₃;
R¹ is a 5-membered heteroaryl group selected from 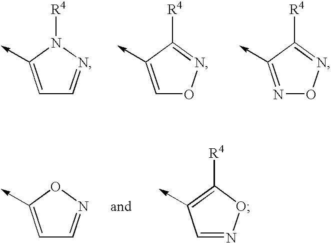
wherein→indicates the point of attachment to the carbonyl moiety; and
R⁴ is hydrogen, (C₁-C₄)alkyl, halo(C₁-C₄)alkyl, or (C₁-C₄)alkoxy(C₁-C₄)alkyl.

2. A compound according to claim 1, or a pharmaceutically acceptable salt or solvate thereof, wherein R¹ is 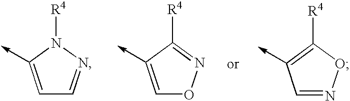
and R⁴ is as defined in claim 1.

3. A compound according to claim 1 or claim 2, or a pharmaceutically acceptable salt or solvate thereof, wherein R⁴ is (C₁-C₄)alkyl, halo(C₁-C₄)alkyl, or (Cᵣ C₄)alkoxy(C₁-C₄)alkyl.

4. A compound according to any one of claims 1 to 3, or a pharmaceutically acceptable salt or solvate thereof, wherein each R² is independently halo or (C₁-C₄)alkoxy.

5. A compound according to any one of claims 1 to 4, or a pharmaceutically acceptable salt or solvate thereof, wherein n is 0, 1, 2 or 3.

6. A compound according to any one of claims 1 to 5 which is selected from:
N-{6-Amino-5-[2-(trifluoromethoxy)phenyl]pyrazin-2-yl}-3-methylisoxazole-4-carboxamide;
N-{6-Amino-5-[2-(trifluoromethyl)phenyl]pyrazin-2-yl}-3-methylisoxazole-4-carboxamide;
N-{6-Amino-5-[5-chloro-2-(trifluoromethoxy)phenyl]pyrazin-2-yl}-3-methylisoxazole-4-carboxamide;
N-{6-Amino-5-[2-(trifluoromethoxy)phenyl]pyrazin-2-yl}-1-ethyl-1H-pyrazole-5-carboxamide;
N-{6-Amino-5-[2-(trifluoromethoxy)phenyl]pyrazin-2-yl}-1-isopropyl-1H-pyrazole-5-carboxamide;
N-{6-Amino-5-[2-(trifluoromethoxy)phenyl]pyrazin-2-yl}-3-(methoxymethyl)isoxazole-4-carboxamide;
N-{6-Amino-5-[5-chloro-2-(trifluoromethoxy)phenyl]pyrazin-2-yl}-3-(methoxymethyl)isoxazole-4-carboxamide;
N-{6-Amino-5-[5-fluoro-2-(trifluoromethyl)phenyl]pyrazin-2-yl}-3-(methoxymethyl)isoxazole-4-carboxamide;
N-{6-Amino-5-[2-(trifluoromethoxy)phenyl]pyrazin-2-yl}-5-(methoxymethyl)isoxazole-4-carboxamide;
N-{6-amino-5-[5-fluoro-2-(trifluoromethyl)phenyl]pyrazin-2-yl}-3-methylisoxazole-4-carboxamide;
N-{6-Amino-5-[5-chloro-2-(trifluoromethoxy)phenyl]pyrazin-2-yl}-1-methyl-1H-pyrazole-5-carboxamide;
N-{6-Amino-5-[2-(trifluoromethoxy)phenyl]pyrazin-2-yl}-3-(trifluoromethyl)isoxazole-4-carboxamide;
N-{6-Amino-5-[5-fluoro-2-(trifluoromethoxy)phenyl]pyrazin-2-yl}-3-methylisoxazole-4-carboxamide;
N-{6-Amino-5-[5-fluoro-2-(trifluoromethyl)phenyl]pyrazin-2-yl}-3-trifluoromethyl)isoxazole-4-carboxamide;
N-{6-Amino-5-[5-fluoro-2-(trifluoromethoxy)phenyl]pyrazin-2-yl}-1-methyl-1H-1-pyrazole-5-carboxamide;
N-{6-Amino-5-[5-ethoxy-2-(trifluoromethoxy)phenyl]pyrazin-2-yl}-1-methyl-1H-pyrazole-5-carboxamide;
N-{6-Amino-5-[5-ethoxy-2-(trifluoromethoxy)phenyl]pyrazin-2-yl}-3-methylisoxazole-4-carboxamide;
N-{6-Amino-5-[5-fluoro-2-(trifluoromethoxy)phenyl]pyrazin-2-yl}-3-(trifluoromethyl)isoxazole-4-carboxamide;
and the pharmaceutically acceptable salts and solvates thereof.

7. A pharmaceutical composition including a compound of the formula (I) or a pharmaceutically acceptable salt or solvate thereof, as defined in any one of claims 1 to 6, together with one or more pharmaceutically acceptable excipients.

8. A compound of the formula (I), or a pharmaceutically acceptable salt or solvate thereof, as defined in any one of claims 1 to 6, for use as a medicament.

9. The use of a compound of the formula (I), or a pharmaceutically acceptable salt or solvate thereof, as defined in any one of claims 1 to 6, for the manufacture of a medicament for the treatment of a disease or condition for which a Na_{V1.8} channel modulator is indicated.

10. Use according to claim 9 wherein the disease or condition is pain.

11. A method of treating a disease or condition for which a Na_{V1.8} channel modulator is indicated in a mammal, including a human, including administering to a mammal requiring such treatment an effective amount of a compound of the formula (I), or a pharmaceutically acceptable salt, solvate or composition thereof, as defined in any one of claims 1 to 6 and 7 respectively.

12. A method according to claim 11 wherein the disease or condition is pain.

13. A compound of the formula (I) or a pharmaceutically acceptable salt or solvate thereof, as defined in any one of claims 1 to 6, for use in the treatment of a disease or condition for which a Na_{V1.8} channel modulator is indicated.

14. A compound of the formula (I) or a pharmaceutically acceptable salt or solvate thereof, as defined in any one of claims 1 to 6, for use in the treatment of pain.

15. A combination of a compound of the formula (I), or a pharmaceutically acceptable salt or solvate thereof, as defined in any one of claims 1 to 8, and another pharmacologically active agent.
